(19) United States
(12) Patent Application Publication
(10) Pub. No.: US 2007/0016163 A1
(43) Pub. Date: Jan. 18, 2007

(54) MEDICAL AND DENTAL IMPLANT DEVICES FOR CONTROLLED DRUG DELIVERY

(75) Inventors: John T. Santini JR., North Chelmsford, MA (US); Charles E. Hutchinson, Canaan, NH (US); Elizabeth R. Proos, Westford, MA (US)

Correspondence Address: SUTHERLAND ASBILL & BRENNAN LLP, 999 PEACHTREE STREET, N.E., ATLANTA, GA, 30309, US

(73) Assignee: MicroCHIPS, Inc., Bedford, MA (US)

(21) Appl. No.: 11/381,646

(22) Filed: May 4, 2006

Related U.S. Application Data
(60) Provisional application No. 60/763,336, filed on Jan. 30, 2006.
(60) Provisional application No. 60/727,323, filed on Oct. 17, 2005.
(60) Provisional application No. 60/695,030, filed on Jun. 28, 2005.

(51) Int. Cl. A61M 31/00

(52) U.S. Cl. 604/500; 424/422; 604/890.1; 623/18.11

(57) ABSTRACT

Implantable devices and methods for use in the treatment of osteonecrosisare provided. The device includes at least one implant device body adapted for insertion into one or more channels or voids in bone tissue; a plurality of discrete reservoirs, which may preferably be microreservoirs, located in the surface of the at least one implant device body; and at least one release system disposed in one or more of the plurality of reservoirs, wherein the release system includes at least one drug selected from the group consisting of bone growth promoters, angiogenesis promoters, analgesics, anesthetics, antibiotics, and combinations thereof. The device body may be formed of a bone graft material, a polymer, a metal, a ceramic, or a combination thereof. The device body may be a monolithic structure, such as one having a cylindrical shape, or it may be in the form of multiple units, such as a plurality of beads.

### CROSS-REFERENCE TO RELATED APPLICATIONS

This application claims benefit under 35 U.S.C. §119(e) of U.S. Provisional Application No. 60/763,336, filed Jan. 30, 2006, U.S. Provisional Application No. 60/727,323, filed Oct. 17, 2005, and U.S. Provisional Application No. 60/695,030, filed Jun. 28, 2005, all of which are incorporated herein by reference.

### BACKGROUND OF THE INVENTION

This invention is generally in the field of implantable medical and dental devices for controlled release of therapeutic and prophylactic agents into a human or animal patient, and particularly prosthetic, drug delivery, or combination implants for replacing, augmenting, or promoting the health of bone, cartilage, or dental tissues.

Hundreds of thousands of hip replacements or revisions are performed each year in the United States. Artificial joints have become a common therapeutic option for replacing the structure of, and restoring function to, injured or diseased joints, including hips, knees, elbows, and shoulders. A few examples of these implantable prosthetic joint devices are described in U.S. Pat. No. 6,436,148, which discloses a joint prosthesis having an overall contour and surface geometry which optimize fixation properties, in U.S. Pat. No. 6,503,281, which discloses a prosthetic assembly for a total hip replacement, and in U.S. Pat. No. 6,945,448 and U.S. Pat. No. 6,797,006, which disclose porous metal orthopedic implants such as femoral knee components or acetabular cups. These patents are incorporated herein by reference.

Risks that may follow the replacement surgery include infection and, in the long term with some types of devices, loss of bone tissue at the interface with the prosthetic device as the bone remodels and consequent loosening of the joint/prosthetic. It would be desirable to deliver one or more drugs locally at the implant site over an extended period of time following implantation of the prosthesis. It would also be desirable to control tissue growth at or into the prosthesis.

Another drawback of joint replacement is that the prosthetic implant eventually will wear out, for example, ten to twenty years following implantation. This is problematic where the patient receiving the joint replacement is relatively young and might be expected to live well beyond the useful life of the joint prosthesis. Replacement of the prosthesis may not be possible in some instances, and would nevertheless require another invasive surgery. It therefore also is desirable to provide implant devices and methods for extending the useful life of a patient's natural bone, joint, or cartilage, so that the need for a complete tissue replacement (e.g., total knee or hip replacement) can be substantially delayed or avoided. Minimization of the effects of wear debris may be exacted by inclusion of a growth factor, such as fibroblast growth factor, in reservoirs on the surface of the implant, an effect which has been demonstrated in a rabbit model. See Shanbhag, et al., “Biological Response to Wear Debris: Cellular Interactions Causing Osteolysis” in The Adult Hip (Callahan, et al., eds.) (Lippincott-Raven Williams, N.Y. 2006).

U.S. Pat. No. 6,799,970, which is incorporated herein by reference, describes a dental implant for anchoring in a bone structure which comprises a head intended to support a dental prosthesis. A significant challenge to the widespread use of dental implants is the often extended time to osseointegration or the growth action of bone tissue, as it assimilates surgically implanted devices or prostheses to be used as either replacement parts (e.g., hip) or as anchors (e.g., endosseous dental implants). It would be desirable to deliver one or more drugs (e.g., a growth factor) locally at the implant site over an extended period of time following implantation to facilitate more rapid or complete osseointegration which may allow for faster time to loading on a new dental prosthesis. It may also be desirable to deliver, independently or concurrently, a localized dose of antibiotic to minimize the risk of infection in or around the implant site, namely at the gingiva.

Certain conventional prosthetic devices may be provided with a coating comprising an antibiotic or growth factor. For example, U.S. Pat. No. 6,736,849, which is incorporated herein by reference, describes a spinal implant prosthetic device, which may be provided with a coating that includes an antibiotic or growth factor. Coatings, however, substantially limit the selection of the coating materials and the drugs, as well as substantially limit the control over the release kinetics and spatial release patterns. Coatings on devices have to be designed for mechanical stability and adhesion, especially when used in locations and device surfaces subject to substantial mechanical loads and/or friction. A particular example of such locations and surfaces are the joints of the skeletal system. Unfortunately, coatings having improved mechanical stability and adhesion may tend to have decreased utility as a controlled drug delivery vehicle. For example, when a coating material is selected that is robust enough not to crack or delaminate from an underlying substrate, it usually performs poorly as a drug delivery vehicle, in that drug does not release as well or as efficiently as would be desired.

It would be desirable to deliver one or more drugs locally at the implant site over an extended period of time following implantation of the prosthesis, to improve control over the release kinetics, and to enable more complex release profiles and patterns, both temporally and spatially.

U.S. Pat. No. 5,947,893, which is incorporated herein by reference, describes a prosthesis having at least one porous tissue-mating surface that includes a coating having a pharmacologically active substance within a biodegradable carrier, such as a polymer or a biodegradable ceramic, such as calcium phosphate. The biodegradable composition of the drug and carrier is impregnated within the pores of the tissue-mating surfaces of the device. Surface coatings, however, are vulnerable to mechanical failure and suffer other limitations. For instance, the choice of coating (drug formulation) material may be limited, because the material needs to be selected to yield a coating having sufficient structural integrity and adhesion properties. Moreover, thin coatings typically provide little actual control over the release kinetics of drugs, due to the extremely short diffusion path of drug from/through the coating. In addition, the use of a thicker coating can result in the creation of gaps between the prosthesis and the patient's tissue after the biodegradable matrix material of the drug formulation has degraded, which undesirably may permit differential motion between the prosthesis and adjacent tissue—and result in undesirable loosening of the prosthetic device. Furthermore, not all drugs are suitable for controlled release from a surface coating, for example, certain drugs, e.g., due to their high aqueous solubility, are released from the coatings at an undesirably high rate and cannot remain localized for a therapeutically effective amount of time. It would be desirable to provide devices and methods for controlling release kinetics of a variety of drugs from implantable prosthetic devices, while avoiding or substantially minimizing the limitations inherent in using a surface coating to modulate drug release.

Osteonecrosis of the femoral head can occur in relatively young people. Approximately 25% of new cases occur in patients younger than 25 years, at an incidence of 10,000-20,000 new cases per year. Many of these cases will require THA (total hip arthroplasty) and account for about 8-12% of the total THAs performed. Given the limited useful lifetime of conventional hip prosthetic implants, it would be highly desirable to delay the need for a total hip replacement for as long as possible in these individuals in particular. Accordingly, there is a need to extend the effective life of the bone tissue (e.g., in a patient exhibiting early stages of osteonecrosis), in particular the tissue of the femoral head.

Initial operative treatments aimed at maintaining the integrity of the femoral head are necessary before a femoral head collapse occurs. This occurs at later stages of osteonecrosis and will ultimately require a total hip replacement. These early treatments include core decompression, osteotomy, nonvascularized bone grafting and vascularized bone grafting. Each technique has limitations. For instance, core decompression has variable success rates with many unduplicated results, osteotomies have low success rates and can complicate future surgical procedures, while bone grafting techniques require the use of autografts or allografts, and in the case of vascularized bone grafts are complex procedures requiring technical expertise and can lead to significant complications. All of these techniques potentially would benefit from the addition of a controlled release of an osteoinductive and/or angiogenic factor for promoting new bone and vasculature formation at the site of the treatment, namely inside the femoral head, by promoting more rapid bone induction/remodeling and by stimulating revascularization in the space left by the removal of the necrotic tissue. More rapid bone formation may allow for faster stabilization and increased strength, potentially leading to shorter recovery periods (including time to unrestricted weight-bearing), better patient compliance, and overall higher success rates. Even where bone formation does not occur faster, such treatments may result in better—e.g., more dense—bone.

It is highly desirable to control infection and/or inflammation at a surgical site, such as the hip or other joint following replacement with a prosthetic implant. A conventional approach includes the use of tethered beads made of a mixture of polymethylmethacrylate (PMMA) and gentamicin, such the product SEPTOPAL™ (Biomet, Inc.). These devices, which can be made as described in U.S. Pat. No. 6,155,812, are used to provide local drug delivery for a period of time following surgery, and later can be retrieved fairly simply. There exists, however, a need to provide more precise control of drug dosing and greater functionality, for example, to deliver multiple drugs with different release rates.

Studies have shown that various anabolic agents including TGF-β may enhance intramembranous bone regeneration, strengthening the mechanical connection between implant and skeleton, which may be necessary for clinical success with orthopedic and dental implants. In addition, plaque accumulation, which leads to inflammatory response, is a primary reason for dental implant failure. It would be desirable to strengthen the mechanical connection between dental implant and skeleton and to prevent pellicle formation. Although the success rate of conventional dental implants is high, implants occasionally fail. It is thus essential for the clinician to identify whether they are ailing, failing, or failed. For this and other reasons, it would desirable to non-invasively assess the status of the implant.

### SUMMARY OF THE INVENTION

Implantable medical devices and methods for use in the treatment of osteonecrosis, as well as in other medical and dental applications, are provided. In one aspect, the device includes at least one implant device body adapted for insertion into one or more channels or voids in bone tissue; a plurality of discrete reservoirs, which may preferably be microreservoirs, located in the surface of the at least one implant device body; and at least one release system disposed in one or more of the plurality of reservoirs, wherein the release system includes at least one drug selected from the group consisting of bone growth promoters, angiogenesis promoters, analgesics, anesthetics, antibiotics, and combinations thereof. The device body may be formed of a bone graft material, a polymer, a metal, a ceramic, or a combination thereof. The device body may be a monolithic structure, such as one having a cylindrical shape, or it may be in the form of multiple units, such as a plurality of beads.

In another aspect, a method for treating osteonecrosis is provided which includes the steps of removing necrotic bone tissue from a bone and creating one or more channels or voids in said bone; and inserting at least one drug delivery device into the one or more channels or voids, wherein the drug delivery device comprises a body portion having a plurality of discrete reservoirs containing at least one release system comprising one or more therapeutic or prophylactic agents for release in vivo. In one embodiment, two or more drug delivery devices are inserted into two or more channels formed in the bone. The channels may be separate and may be parallel. In another embodiment, the method further includes utilizing a fluid delivery means to wet the drug delivery device which is disposed in the one or more channels or voids. For example, the fluid delivery means may be a re-routed or grafted blood vessel, or it may be include a fluid source, pump, and at least one catheter, wherein the distal end of the catheter is inserted into at least one of the channels or voids containing the drug delivery device and delivers fluid from the fluid source via the pump. The fluid reservoir and pump may be integrated into a single device. The fluid source may be saline, blood, or a blood component, and may include hyaluronic acid. In one embodiment of the method, the step of removing necrotic bone tissue from a bone and creating one or more channels or voids in said bone may involve a light bulb or trapdoor surgical procedure.

In another aspect, a joint resurfacing device is provided that includes a body portion having a joint tissue interfacing surface and an opposing side; a plurality of discrete reservoirs located joint tissue interfacing surface; at least one release system disposed in one or more of the plurality of reservoirs containing at least one release system comprising one or more therapeutic or prophylactic agents for release in vivo; and an anchor portion extending from the opposing side away from the joint tissue interfacing surface, wherein the anchoring portion is adapted to secure the joint resurfacing device to a bone in need of resurfacing. The one or more therapeutic or prophylactic agents may be a bone growth promoter or may be selected from bone morphogenic proteins, angiogenesis promoters, analgesics, anesthetics, antibiotics, and combinations thereof. In one embodiment, the joint tissue interfacing surface comprises a rounded cap. In one embodiment, the reservoirs have chamfered openings in the surface of the joint tissue interfacing surface. The anchor portion may include on one or more screws.

In still another aspect, an implantable infection control device is provided, which includes a plurality of beads tethered together to form a chain, wherein the beads comprise a plurality of discrete reservoirs which are loaded with a release system comprising at least one anti-infective drug formulation for controlled release in vivo. The beads may be cylindrical, spherical, or elliptical shaped, and typically are made of a biocompatible material selected from polytetrafluoroethylenes, polyesters, polymethylmethacrylates, silicones, metals, glasses, ceramics, bone cements, and combinations thereof. In one embodiment, the release system includes at least one antibiotic agent dispersed in a polymeric matrix material. In one embodiment, the beads are tethered by at least one biocompatible string imbedded through the beads or threaded through apertures in the beads. In one embodiment, at least one of the beads comprises a first drug and at least another of the beads comprises a second, different drug.

In various embodiments of the beads device, a first group of the reservoirs comprises the at least one anti-infective drug formulation and a second group of the reservoirs comprises a second formulation of a drug, wherein the at least one anti-infective drug formulation and the second formulation have different compositions. In one case, the drug of the at least one anti-infective drug formulation is different from the drug of the second formulation. The second formulation of a drug may include an anti-inflammatory agent. The device may be adapted to provide simultaneous release of the two or more drugs, or, in one embodiment, the release system is layered to provide serial release of two or more drugs.

In a further aspect, a prosthetic dental device is provided which includes a device body having an anchor portion adapted for engagement with a jaw bone of a patient in need thereof; two or more discrete reservoirs, which may preferably be microreservoirs, that are located in spaced apart positions in the device body, the reservoirs formed with an opening at the surface of the device body and extending into the device body; and a release system disposed in the reservoirs which comprises at least one therapeutic or prophylactic agent, wherein following implantation into a patient the therapeutic or prophylactic agent is released in a controlled manner from the reservoirs. The device may include a replacement tooth portion. The reservoirs may be located in the anchor portion of the device body. In one embodiment, the device body includes a stainless steel, a chrome-cobalt alloy, a titanium alloy, a ceramic, an ultra high molecular weight polyethylene, or a combination thereof. The anchor portion may include at least one screw-like, threaded region. The therapeutic or prophylactic agent may include one or more anti-infective, antibiotic agents, growth factors, or a combination thereof In one embodiment, the device is adapted to release two or more, different therapeutic or prophylactic agents. In an example, one of the therapeutic or prophylactic agents is disposed in a first array of reservoirs and a second of the therapeutic or prophylactic agents is disposed in a second array of reservoirs. The first array may be located to release one or more anti-infective or antibiotic agents into gum tissues, and the second array may be located to release one or more growth factors into orthopedic tissues.

In yet another aspect, a prosthetic dental device is provided that includes a device body having an anchor portion for engagement with a jaw bone of a patient in need thereof; and at least one sensor or diagnostic agent integrated into the device body. The sensor may be used to measure temperature, pressure, or both in one or more areas in or around the site of in vivo implantation of the device.

### BRIEF DESCRIPTION OF THE DRAWINGS

FIG. 1 is a perspective view and magnified view of one embodiment of hip prosthetic device that includes reservoirs for passive, controlled drug delivery.

FIGS. 2A-D are cross-sectional views of various embodiments of a prosthetic device body surface that includes regions of porosity and discrete reservoirs.

FIG. 3 is a plan view of one embodiment of a device which includes a tube which has a plurality of drug-containing reservoirs for active, controlled release of drug, for delivering drug into bone joints and other small spaces.

FIGS. 4A-C are plan (4A) and cross-sectional views (end on cross-section, 4B and side on cross-section, 4C) of one embodiment of the tip of the tube of the device shown in FIG. 3.

FIGS. 5A-C are perspective (5A) cross-sectional views (interior view 5B and end on cross-section, 5C) of one embodiment of a spinal cage prosthetic device.

FIG. 6 is a perspective view of one embodiment of a device for infection control, which controllably releases drug locally at a site of implantation.

FIGS. 7A-C illustrate one embodiment of a device and method for the treatment of osteonecrosis of the femoral head. FIGS. 7A and 7B are a perspective view and a cross-sectional view of the drug delivery device adapted to fit into a channel in the femoral head as shown in the perspective, cross-section view of FIG. 7C.

FIGS. 8A-B illustrate another embodiment of a device and method for the treatment of osteonecrosis of the femoral head using multiple channels. FIG. 8A is a perspective view of a drug delivery device, a plurality of which is adapted to fit into channels in the femoral head as shown in the perspective, cross-section view of FIG. 8B.

FIG. 9 is a perspective and cross-sectional view of one embodiment of a technique for the treatment of osteonecrosis of the femoral head, wherein a fluid delivery system is used in combination with a drug delivery device implanted into a channel in the femoral head.

FIGS. 10A-D illustrate another embodiment of materials and methods for the treatment of osteonecrosis of the femoral head, wherein the “light bulb” procedure is used in combination with beads which include reservoirs for controlled drug delivery. FIGS. 10A-B are perspective views of the beads, FIG. 10C is a cross-section view of one of the beads, and FIG. 10D is a perspective, cross-sectional view of the femoral head following the beads implantation.

FIGS. 11A-B illustrate another embodiment of a device and method for the treatment of osteonecrosis of the femoral head, wherein the “light bulb” procedure is used in combination with a single implant device which includes reservoirs for controlled drug delivery. FIG. 11A is perspective view of the device, and FIG. 11B is a perspective, cross-sectional view of the femoral head following the device implantation.

FIGS. 12A-D illustrate another embodiment of materials and methods for the treatment of osteonecrosis of the femoral head, wherein the “trap door” procedure is used in combination with beads or a single structure which include reservoirs for controlled drug delivery. FIGS. 12A-B are perspective views of the opened debrided femoral head site and the same site following completion of procedure. FIG. 12C and FIG. 12D are cross-sectional views of the implanted beads and implanted device, respectively.

FIG. 13 is a cross-sectional view, with portion magnified, of one embodiment of a joint resurfacing device which includes reservoirs for controlled drug delivery.

FIG. 14 is a plan and partial cross-sectional view of one embodiment of an implanted prosthetic dental device that has reservoirs for controlled local drug delivery. The anchor portion is in plan view, with the replacement tooth, mounting portion, gum, and bone in cross-section.

FIG. 15 is a plan and partial cross-sectional view of another embodiment of an implanted prosthetic dental device that has reservoirs for controlled local drug delivery. The anchor portion is in plan view, with the replacement tooth, mounting portion, gum, and bone in cross-section.

FIG. 16 is a plan and partial cross-sectional view of another embodiment of an implanted prosthetic dental device that has reservoirs for controlled local drug delivery located on the anchor portion of the device body. The anchor portion is in plan view, with the replacement tooth, gum, and bone in cross-section.

### DETAILED DESCRIPTION OF THE PREFERRED EMBODIMENTS

Implantable devices and methods of use have been developed to provide controlled delivery of therapeutic and prophylactic agents in the treatment and health of orthopedic, joint, spinal, and dental tissues. As used herein, the term “orthopedic” includes and is synonymous with the term “orthopaedic.” In one particular aspect, the devices and methods are used in the treatment of avascular necrosis, providing improved controlled delivery of bone growth promoters and other drugs directly where needed. As used herein, the terms “avascular necrosis” and osteonecrosis” are synonymous and may be used interchangeably.

In another aspect, the device is a prosthetic device. As used herein, the term “prosthetic” refers to medical and dental devices that are primarily used to secure together separate tissue portions or to provide a load bearing function. It is considered prosthetic in the sense that it is serving as a structural complement or substitute (permanently or temporarily) for one or more tissues of the body, particularly hard tissues.

In a preferred embodiment, the device includes an array of discrete reservoirs (at least two, and more preferably hundreds), particularly microreservoirs, that are provided across one or more outer surface areas of the device body. Tese reservoirs contain a release system comprising one or more therapeutic or prophylactic agents formulated to provide a desired level of drug stability, and the release system, formulation, and/or reservoir caps control the time and rate of release of the agent in vivo following implantation. By containing the drug and controlled release formulation within discrete reservoirs built into (at least a portion of) the structure of the device body, one advantageously can avoid certain limitations that would otherwise have been obtained by use of a surface coating of the drug formulation, while enabling sustained or controlled drug release in complex temporal or spatial release profiles. For instance, one can use a desired drug formulation that might not have the mechanical strength properties needed for the drug formulation to be used as a surface coating on a prosthetic device body, but that works well when stored in discrete reservoirs located in a surface of the prosthetic device body.

In another embodiment, the drug helps minimize the risk of prosthetic joint infection or other site-specific infection due to implantation of an orthopedic, spine, or dental device. For example, the device can release a therapeutic or prophylactic effective amount of one or more antibiotics (e.g., cefazolin, cephalosporin, gentamycin, tobramycin, etc.) and/or another agent effective in preventing or mitigating biofilms (e.g., a quorum-sensing blocker or other agent targeting biofilm integrity). Bacteria tend to form biofilms on the surface of implant devices, and these biofilms, which are essentially a microbial ecosystem with a protective barrier, are relatively impermeable to antibiotics. Accordingly, systemically administered antibiotics may not achieve optimal dosing where it is needed most. However, the present devices enable the delivery of the desired dose of antibiotic precisely when and precisely where needed—in particular beneath the biofilm. In addition, the device can be designed to release the drug in various temporal and spatial patterns/profiles, e.g., releasing drug in a continuous or pulsatile manner for several days and/or targeting areas of the device, if any, that are more conducive to bacterial growth. For example, the device could release an antibiotic over a period of 5 to 15 days, which would be a typical antibiotic treatment regimen. In one embodiment, revision implants are provided with reservoirs on the implant surface or in crevices or channels, which are loaded with a stable antibiotic formulation with optimized release kinetics. In this way, the antibiotic agent can be released under a bacterial biofilm that may form from bacteria harbored in crevices of a prosthetic implant. The local delivery of antibiotic agents can decrease undesirable systemic drug exposure (and deleterious side effects caused thereby). In another embodiment, following a total knee replacement, the prosthetic knee device includes a plurality of discrete reservoirs for releasing an antibiotic or other drug.

In a preferred embodiment, the present drug-eluting device is adapted for use in the treatment of cancer of the bone or joint. For example, osteosarcoma or chondrosarcoma often are treated surgically by excision requiring removal of significant amounts of bone and soft tissue. Care must be taken to avoid spilling the tumor during resection to avoid seeding of tumor cells into surrounding tissues. It therefore would be beneficial for the prosthetic implant to release one or more local chemotherapeutic agents into the surrounding tissue following implantation, in order to destroy tumor cells remaining at the surgical site following resection, to complement or replace the systemic chemotherapy and/or radiation therapy that typically is prescribed for the patient. In variations of these embodiments, the implant device releases one or a combination of therapeutic agents, including chemotherapeutic agents (e.g., paclitaxel, vincristine, ifosfamide, dacttinomycin, doxorubicin, cyclophosphamide, fluorouracil, carmustine, and the like), cytokines (e.g., IL-2, interferon-α2b), bisphosphonates (e.g., pamidronate, clodronate, zoledronic acid, and ibandronic acid), analgesics (such as opoids and NSAIDS), anesthetics (e.g., ketoamine, bupivacaine and ropivacaine), tramadol, and dexamethasone. In one embodiment, the implant device releases carmustine (BCNU), alone or in combination with interleukin-2, which may preferably be released at different times. See, e.g., Rhines et al., “Local Immunotherapy with Interluekin-2 Delivered from Biodegradable Polymer Microspheres Combined with Interstitial Chemotherapy,” 52 (4):872-880 Neurosurgery (April 2003); Sampath, et al., Cancer Research 59:2107-114 (1991), which demonstrate the potential benefit of high dose, local chemotherapy.

In another embodiment, the drug-eluting device possesses a two-fold treatment achieved via two therapeutic formulations with temporally different release profiles: A first one delivering chemotherapeutic treatment immediately after resection to ensure complete inactivation of any remaining tumor cells and second one delivered at some later time point (days or weeks, etc.) that delivers one or more growth factors or other therapeutic agents to promote healing and bridge the bone gap left by the resection. The release profiles of the two therapeutic formulations also may differ spatially, releasing the drugs from reservoirs located in different areas of the device. In a further variation of this embodiment, a local analgesic or anesthetic may be controllably released from the device (e.g., from another set of reservoirs over the entire healing period. This embodiment may be particularly beneficial because osteosarcomas are painful tumors that often can occur in a young patient population.

In another embodiment, the drug facilitates vascularization at or into the implanted prosthetic device or promotes bone health and growth. For example, the drug can be a bone morphogenic protein (BMP) or recombinant version thereof (rBMP), which facilitates bone formation around or, in the case of a device having a porous surface, into the implanted prosthetic device. Representative examples of BMPs include BMP-2, -3, -4, -7, and -9, where rhBMP-2 and rhBMP-7 may be preferred. Other examples of drugs for promoting new vascularity include a fibroblast growth factor (FGF), such as FGF-2 or VEGF. This could be particularly desirable where the prosthesis is secured without the use of cement, although it could possibly be used in combination with cement.

In a preferred embodiment, the drug is used in the management of pain and swelling following the implantation surgery. For example, the device can release an effective amount of an analgesic agent alone or in combination with an anesthetic agent. In another case, the device can release an anti-inflammatory agent, alone or in combination with an analgesic agent or an anesthetic agent.

In several preferred embodiments, the device releases a combination of different substances to improve bone healing. For example, the device can release different combinations of growth factors (e.g., (TGF)-β, BMP, VEGF, FGF, IGF, GDF, PDGF, PTH), osteoinductive molecules, hormones, anti-TNF (tumor necrosis factor) agents, bone resorption inhibitors (e.g., bisphosphonate), and bone-forming cells (e.g., osteoblasts, adult stem cells, osteoprogenitor cells). These different molecules and cells can be delivered at varied spatial positions and temporal sequences during bone healing. In one particular embodiment for the repair of local bone erosions, which often are associated with rheumatoid arthritis, the prosthetic device locally delivers (1) an anti-TNF agent, which reduces inflammation that fuels bone erosion, and (2) parathyroid hormone (PTH), which stimulates bone formation, and/or osteoprotegrin (OPG), which blocks bone resorption and can lead to repair of local bone erosions and reversal of systemic bone loss. Examples of anti-agents include TNF antagonists, such as etanercept (Enbrel™, Amgen and Wyeth) and infliximab (Remicade™, Centocor), which have shown efficacy and have been approved by the U.S. FDA for the treatment of rheumatoid arthritis.

In yet another embodiment, the drug can be one selected to mitigate the risk of formation of blood clots at the implant site, which can lead to venous thromboembolism or pulmonary embolism. For instance, the device may be used to release one or more anticoagulants and/or antiplatlet drugs (e.g., heparins, aspirin, clopidogrel, lepirudin, fondaparinux, warfarins, dicumarol, etc.).

In still a further embodiment, the drug stored in and released from the reservoirs is a self-propagating agent, such as a gene therapy agent or vector. A desired local or systemic response is created following release of the small amount of agent.

Representative examples of therapeutic or prophylactic agents that may be released from the prosthetic device include analgesics, anesthetics, antimicrobial agents, antibodies, anticoagulants, antifibrinolytic agents, anti-inflammatory agents, antiparasitic agents, antiviral agents, cytokines, cytotoxins or cell proliferation inhibiting agents, chemotherapeutic agents, hormones, interferons, and combinations thereof. In one embodiment, the device provides for the controlled release of a growth factor, such fibroblast growth factors, platelet-derived growth factors, insulin-like growth factors, epidermal growth factors, transforming growth factors, cartilage-inducing factors, osteoid-inducing factors, osteogenin and other bone growth factors, and collagen growth factors. In another embodiment, the device provides for controlled release of a neutrophic factor (which may be of benefit in spinal prosthetic applications) or a neutropic factor. In one embodiment, the drug is a tumor necrosis factor.

In one embodiment, the drug is in an encapsulated form. For example, the drug can be provided in microspheres or liposomes for controlled release. In another embodiment, the drug is provided in nanoparticle form.

Preferably, release of the drug is passively controlled. However, the prosthetic device body can, alternatively or further, include active mechanisms for controlling release from reservoirs. The active control and/or power mechanisms could, for example, be attached to or imbedded within a surface of the prosthetic device, or could be built into inside (e.g., in an interior space of) the prosthetic device.

Illustrative Embodiments of the Drug-Eluting Devices

In one aspect, an implantable device, preferably a prosthetic device, for controlled drug delivery is provided which includes: a prosthetic device body having at least one outer surface area; two or more discrete reservoirs located in spaced apart positions across at least a portion of the outer surface area, the reservoirs being formed with an opening at the surface of the device body and extending into the device body; a release system disposed in the reservoirs which comprises at least one therapeutic or prophylactic agent, wherein following implantation into a patient the therapeutic or prophylactic agent is released in a controlled manner, at effective rates/times, from the reservoirs.

Device Body

In various embodiments, the device body may be a joint (e.g., knee or hip) or bone prosthesis or part thereof, a spinal fusion cage or spinal disk prosthesis, a trauma or fixation device, or a dental or maxillofacial prosthetic device. The device body may be formed of a biocompatible metal, a ceramic (e.g., a phosphate) a polymer, or a combination thereof. The device includes an array of discrete reservoirs in one or more surfaces, which are loaded with a release system comprising one or more therapeutic or prophylactic agents for controlled release. Such reservoirs could be loaded with a stable OP-1 (i.e., BMP-7) formulation with optimised release kinetics and optionally loaded with an antibiotic agent for biofllm control. These or other reservoirs could be sized and located to enhance device fixation, e.g., by promoting osteointegration. In a preferred variation, the reservoirs of the device release one or more anti-infective agents.

In a preferred embodiment, the device body is substantially rigid, with a defined geometry. That is, it is not a spongy or putty-like material that takes the shape of the space in which it is implanted.

In preferred embodiments, the device body and surface area in which the reservoirs are defined can be formed of, be coated with, or otherwise comprise a biocompatible material selected from metals, polymers, ceramics, and combinations thereof. Typically, the device body is non-biodegradable, as the prosthetic device is intended to last for an extended period of time, preferably for the life of the patient. For instance, the device body can comprise a stainless steel, a chrome-cobalt alloy, a titanium alloy, pure titanium (as is the case for dental implants), tantalum or porous tantalum (e.g., TRABECULAR METAL™ (Zimmer)), oxidized zirconium (e.g., OXINIUM™ (SMITH & NEPHEW)), a ceramic, or an ultra high molecular weight polyethylene. In other embodiments, the device body is formed of or includes a ceramic (e.g., alumina, silicon nitride, zirconium oxide, various carbides), a semiconductor (e.g., silicon), a glass (e.g., Pyrex™, BPSG), or a degradable or non-degradable polymer.

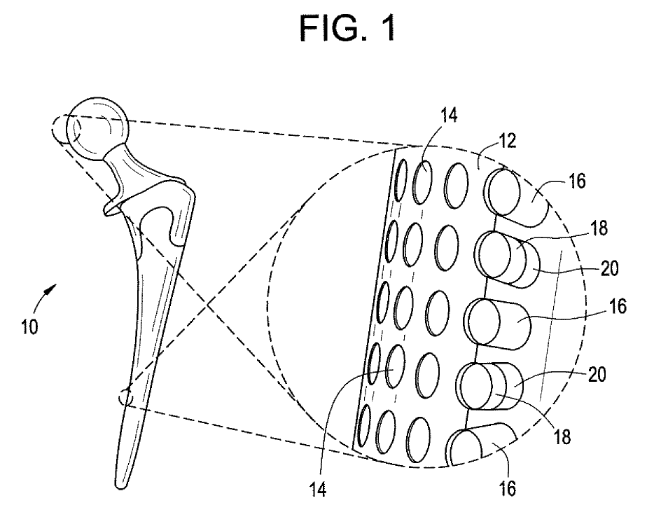

One embodiment of a hip prosthetic device is shown in FIG. 1, which illustrates the ball and stem portion 10 of a hip prosthesis. The device body has an outer surface 12 which includes an array of reservoirs 14 disposed therein. In this particular embodiment, select reservoirs contain a first drug formulation 16, and select other reservoirs contain a second drug formulation 20. The reservoirs of the second drug formulation include reservoir caps 18 covering the second drug formulation 20. Alternatively, reservoir caps 18 could be replaced with another drug formulation, such that layers 18 and 20 would provide a reservoir filled with layers of two different drugs or two different dosages/formulations of a single drug. The two layers could be formulated to provide different release rates of the same drug.

In one example, the total hip prosthesis consists of three parts: (1) a metal cup (called the acetabulum or acetabular component) that replaces the hip socket, which cup has a liner made of a polymer (e.g., a high molecular weight polyethylene), ceramic, or metal material; (2) a metal or ceramic ball that replaces the damaged head of the femur; and (3) a metal stem that is inserted into or attached to the shaft of the bone to add stability to the prosthesis. The reservoirs can be provided on any or all surfaces of such a prosthesis.

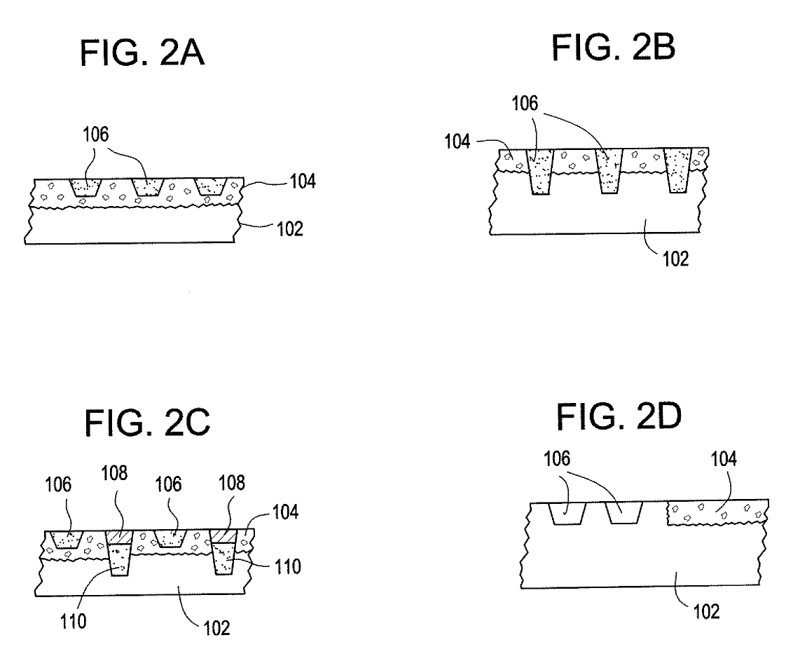

The surface of the device body where the reservoirs are located can be porous or non-porous. Optimal bony-ingrowth is expected to be provided into prosthesis devices that include pores of approximately 250 to 500 microns. In one embodiment, the entire surface of the device is porous. In another embodiment, a portion, e.g., a portion of the tissue- or bone-mating surfaces, of the prosthesis is porous, to provide at least one tissue-contact surface that provides stable fixation in the body. FIGS. 2A-C illustrate some of the various combinations of porous and non-porous substrate (body) materials with different reservoirs. FIG. 2A shows a portion of a device body having non-porous region 102 with porous surface region 104, in which discrete reservoirs are disposed in spaced positions (i.e., in an array). The reservoirs are filled with drug formulation 106, such as drug dispersed in a soluble or biodegradable matrix material, such as biocompatible polymer, e.g., PLGA, PGA, PLA, PEG, or various poly(anhydrides). In this embodiment, the reservoirs are located only in the porous region. In contrast, FIG. 2B show a device in which the reservoirs extend into the non-porous region. In FIG. 2C, some reservoirs are shallower and some are deeper, such that only the deeper ones extend into the non-porous region. In this embodiment, the shallower reservoirs contain a first drug formulation 106, and the deeper reservoirs are filled with two or more distinct layers: An outer layer 108, which can be formed of one or more non-bioactive materials (e.g., a biodegradable, protective reservoir cap) that can delay exposure of an inner layer 110, which can comprise a drug—the same as or different from the drug in formulation 106. FIG. 2D illustrates an embodiment having a surface comprising both porous and non-porous regions. The non-porous region 102 includes reservoirs containing drug formulation 106, and the porous region 104 may, for example, be selected to have a porosity that facilitates tissue ingrowth. In one embodiment, the device body includes or consists of a completely porous material, such as a trabecular metal, e.g., tantalum (which may be provided by Zimmer Technology, Inc.). In one embodiment, the drug released from the reservoirs next to the porous region could be a growth factor to enhance bone growth in to the porous region or an antibiotic to prevent infection. Other variations and combinations of these embodiments are envisioned.

Optionally, the device body may be installed into the bone site with a biocompatible cement. The surface of the device body to be cemented can be porous or non-porous. Examples of biocompatible cements known in the art include polymethylmethacrylates (PMMAs) and PALACOS™ (Heraeus Kulzer, Germany). In preferred embodiments, the reservoirs are positioned away from the area(s) that are cemented, so as not to impede or interfere with release of the drug(s) from the reservoirs.

The shape of the device body depends on the particular application. The device body preferably is a rigid, non-degradable structure. The body may consist of only one material, or may be a composite or multi-laminate material, that is, composed of several layers of the same or different substrate materials that are bonded together. In another embodiment, the device body is not actually a prosthetic but is used in the treatment of an orthopedic disease or disorder.

In one embodiment, a method is provided for local delivery of a therapeutic or prophylactic agent in the treatment of difficult to access orthopedic tissues, such as joint spaces. This is particularly wherein active control of drug release is desired, but there is little or no space for a larger implant device with associated electronics and power sources. In one case, the method includes implanting at a orthopedic tissue site, such as a joint or spinal disc, a tip portion of a tube which comprises a first end and a distal second end, wherein the tip portion has located therein a plurality of discrete reservoirs containing a therapeutic or prophylactic agent, the reservoirs having openings sealed by a plurality of discrete reservoir caps; and actively and selectively disintegrating the reservoir caps to initiate release of the therapeutic or prophylactic agent at the tissue site.

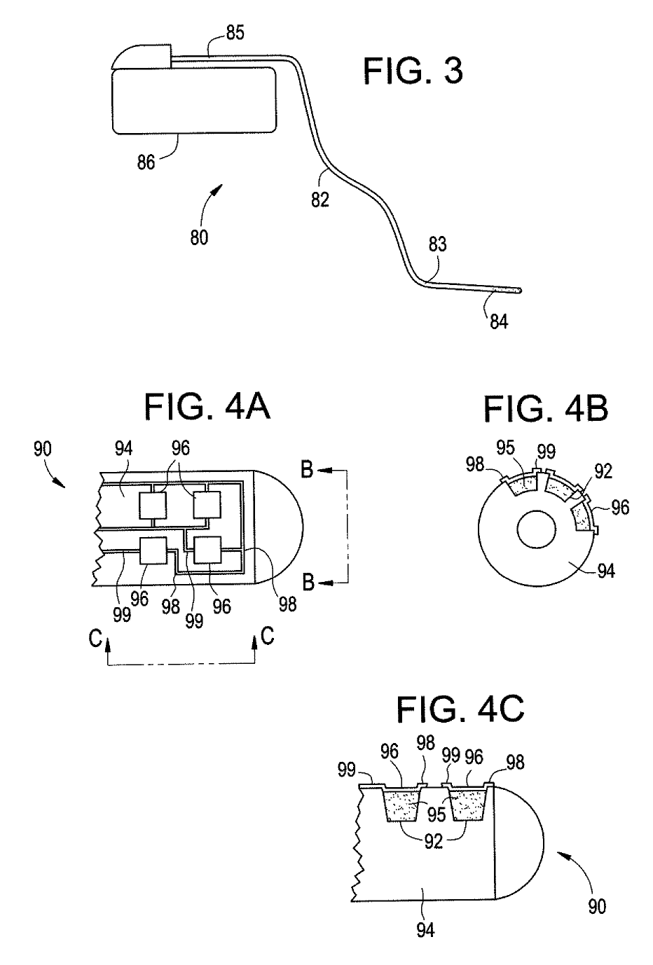

In one embodiment of an active device, which is illustrated in FIG. 3, the implantable device 80 includes a tube 82, which has a plurality of drug-containing reservoirs 84 fabricated at the tip portion 83 of the tube. (The “tube” may sometimes be referred to in the art as a “catheter.”) The tube tip can be made of biocompatible metal, ceramic, silicon, or polymer, and it serves as the substrate/device body in which the discrete reservoirs are fabricated and arrayed.

The power source and control hardware 86 are located at the proximal end of the catheter 85, so they need not fit into or be located at the delivery site. The tube includes wires/electrical connections for connecting electronics in the tip portion. In one embodiment, the tip portion of the tube includes tens or hundreds of micro-reservoirs containing a drug formulation and hermetically sealed by conductive reservoir caps. FIGS. 4A-C illustrates one embodiment of the tube tip portion 90 which has reservoirs 92 in substrate/ tube body 94, wherein the reservoirs contain therapeutic agent 95 and are covered by conductive reservoir caps 96, each of which are connected to input and output electrical leads 98 and 99, respectively. The reservoir caps are electrically connected to a power source and can be disintegrated by electrothermal ablation as described in U.S. Patent Application Publication No. 2004/0121486 A1. Alternatively, the reservoir caps may be activated (e.g., disintegrated) by another mechanism, such as electrochemically, thermally, etc. as disclosed in U.S. Pat. No. 5,797,898, U.S. Pat. No. 6,527,762, U.S. Pat. No. 6,571,125, which are incorporated herein by reference.

The power source and control hardware can be surgically placed in a subcutaneous pocket under the intraclavicular fossa or in the abdominal wall, and the catheter extending therefrom can be threaded into the therapeutically desirable location at a vertebrae, at the brainstem, in an intrathecal space, near an organ or the heart, or near or on another select tissue structure. Alternatively, the power/control unit can be externally worn and provided with a catheter through the patient's skin. The electrical traces could be built into the catheter body or supported on an inner or outer surface of the catheter body. The tube tip can be made of biocompatible metal, ceramic, or polymer, and it serves as the substrate in which the discrete reservoirs are fabricated and arrayed. In one embodiment, the tube is replaceable and removably secured to the power/control unit, so that when all of the reservoirs are depleted of drug, then the catheter can be replaced with a minimally invasive procedure, since the power/control unit need not be replaced as frequently, if at all. The implanted power/control unit can be battery powered and pre-programmed or wirelessly powered and wirelessly controlled externally. The tip also may be placed in or near joints where a larger device could not fit. For example, the tip may be placed in the intercondylar fossa in the knee joint to release anti-infectives or anti-inflammatory drugs. The power source and control electronics could be placed under the skin in the thigh or in the abdomen.

In an alternative embodiment (not shown) drug release from the plurality of discrete reservoirs is passively controlled. The tube may have a solid core or could have a central, longitudinal aperture for delivering a fluid therethrough.

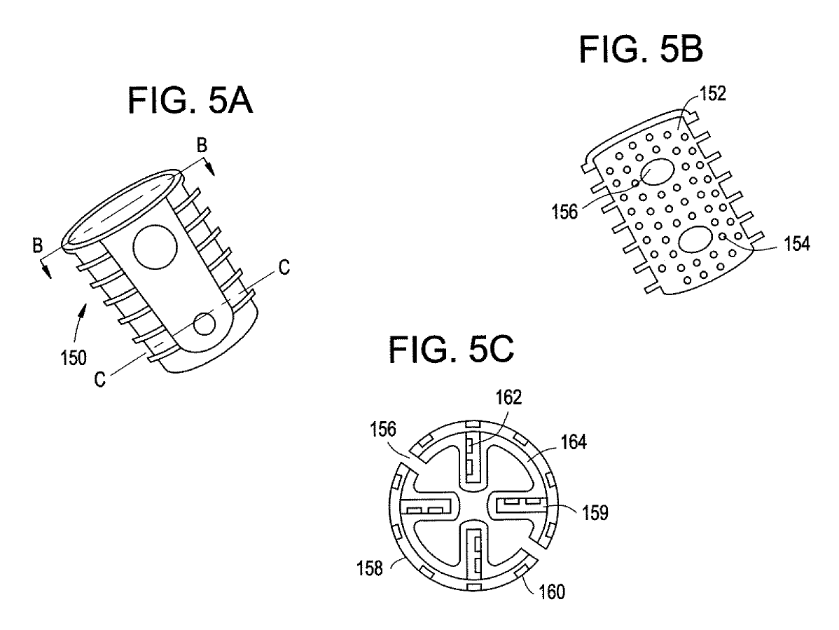

One embodiment of a lumbar tapered fusion prosthetic device is shown in FIGS. 5A-C. Device 150 includes interior surface 152 in which interior reservoirs 154 are disposed. The device body includes sidewall 158 that has exterior reservoirs 160 and major apertures 156, which are provided for bone to grow into/through the device to lock it into place, providing a bridge of bone extending from one vertebra to the next. The interior of the device includes baffles 159, which are coated with a tissue scaffolding material 164, such as a hydrogel. The baffles also include baffle reservoirs 162. For clarity, exterior reservoirs 160 are not show in view FIG. 5A.

Reservoirs

The reservoirs are located in spaced apart positions across one or more areas of the surface of the device body. The reservoirs are formed with an opening at the surface of the device body and extend into, or through, the device body. As used herein, the term “reservoir” means a well, a cavity, or a hole suitable for storing, containing, and releasing/exposing a precise quantity of a material, such as a drug formulation. In preferred embodiments, the reservoirs are discrete, non-deformable, and disposed in an array across one or more surfaces (or areas thereof) of the device body. The device body preferably has many reservoirs. In various embodiments, tens, hundreds, or thousands of reservoirs are arrayed across the device body.

The interconnected pores of a porous material are not reservoirs. Pores are not considered reservoirs, because of their random nature (random in size, shape, and location), which renders them unsuitable for controlling release kinetics. That is, one cannot accurately know the amount of drug and the spatial homogeneity of the drug formulation contained within a porous material, so the control of the release kinetics is much more difficult.

The reservoirs can be fabricated into the device body by any of a number of methods and techniques known in the art, depending on various parameters including the materials of construction of the device body, the dimensions of the reservoirs, the location of the reservoirs on the device body, and the shape and configuration of the device body. Reservoirs may be created in the device body simultaneously with formation of the device body, or they may be formed in the device body after the device body is made. Accordingly, the reservoirs can be made by a variety of techniques, including MEMS fabrication processes, microfabrication processes, or other micromachining processes, various drilling techniques (e.g., laser, mechanical, and ultrasonic drilling), electrical discharge machining (EDM), and build-up or lamination techniques, such as LTCC (low temperature co-fired ceramics) and sand, grit, and other particle blasting techniques. Numerous other methods known in the art can also be used to form the reservoirs. See, for example, U.S. Pat. No. 6,123,861 and U.S. Pat. No. 6,808,522. Microfabrication methods include lithography and etching, injection molding and hot embossing, electroforming/electroplating, microdrilling (e.g., laser drilling), micromilling, electrical discharge machining (EDM), photopolymerization, surface micromachining, high-aspect ratio methods (e.g., LIGA), micro stereo lithography, silicon micromachining, rapid prototyping, and DEEMO (Dry Etching, Electroplating, Molding). Reservoirs may be fabricated into metal body portions by techniques known in the art, including laser etching, laser jet etching, micro-EDM, oxide film laser lithography, and computerized numerical control machining.

In one embodiment, the reservoirs are formed in the substrate by laser drilling, EDM, or other mechanical or physical ablative methods. In another embodiment, the reservoirs are fabricated by a masking and chemical etching process. In embodiments where the device comprises a porous surface, the reservoirs can be fabricated before or after a porosity-inducing step. For instance, reservoirs can be mechanically formed into the porous surface, optionally penetrating into the non-porous region beneath. Alternatively, porosity can be creating in the surface, for example, by a chemical etching process after formation of the reservoirs. In order to preserve the defined boundaries of the reservoirs, the reservoirs can be filled with a temporary fill material, such as a wax, that is resistant to the chemical etch, prior to etching and then the fill material can be removed following etching, for example, by heating and volatilizing the wax or by use of an appropriate solvent selective for the temporary fill material. One process for creating surface microporosity in a titanium or other metal surface is described in U.S. Patent Application Publication No. 2003/0108659 A1 to Bales et al., which is incorporated herein by reference.

The reservoirs may be defined by one or more sidewalls, a bottom wall, an open end (an opening) distal the bottom wall. The opening is at a surface of the device body from which release of the therapeutic or prophylactic agent is desired. In a preferred embodiment, all of the reservoir walls (side and bottom) are non-porous. In another embodiment, a majority of the reservoir walls are non-porous, e.g., where the reservoir extends through a porous surface region (and into a non-porous region) of the device body. In another embodiment, reservoirs may extend through the device body, providing for instance a reservoir having two opposed openings (no bottom wall).

In a preferred embodiment, the reservoirs are microreservoirs. The use of microreservoirs may be particularly beneficial to minimally impact the strength and structural integrity of the device body, as compared to the mechanical property losses that could occur with the use of macroreservoirs. A “microreservoir” is a reservoir having a volume equal to or less than 500 μL (e.g., less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). In another embodiment, the reservoirs are macroreservoirs. A “macroreservoir” is a reservoir having a volume greater than 500 μL (e.g., greater than 600 μL, greater than 750 μL, greater than 900 μL, greater than 1 mL, etc.) and less than 5 mL (e.g., less than 4 mL, less than 3 mL, less than 2 mL, less than 1 mL, etc.). The shape and dimensions of the reservoir, as well as the number of reservoirs, can be selected to control the contact area between the drug material and the surrounding surface of the reservoirs. Unless explicitly indicated to be limited to either micro- or macro-scale volumes/quantities, the term “reservoir” is intended to encompass both. In one embodiment, the device may include a combination of both microreservoirs and macroreservoirs.

Release System and Therapeutic/Prophylactic Agent

The release system comprises at least one therapeutic or prophylactic agent (sometimes referred to herein as a “drug”). The release system is disposed in the reservoirs, so as to be isolated, e.g., protected, from the environment outside of the reservoir until a selected point in time, when its release or exposure is desired. The term “release system,” is as described in U.S. Pat. No. 5,797,898, which is incorporated herein by reference. The therapeutic or prophylactic agent can be dispersed in a matrix material, which by its degradation, dissolution, or diffusion properties provides a means for controlling the release kinetics of the therapeutic or prophylactic agent. The released therapeutic or prophylactic agents are primarily intended for local or regional effect, but may in some embodiments be intended for systemic delivery.

The therapeutic or prophylactic agent can be essentially any active pharmaceutical ingredient, or API. It can be natural or synthetic, organic or inorganic molecules or mixtures thereof. The therapeutic or prophylactic agent molecules can be mixed with other materials to control or enhance the rate and/or time of release from an opened reservoir.

The therapeutic or prophylactic agent molecules may be in essentially any form, such as a pure solid or liquid, a gel or hydrogel, a solution, an emulsion, a slurry, or a suspension. In various embodiments, the therapeutic or prophylactic agent molecules may be in the form of solid mixtures, including amorphous and crystalline mixed powders, monolithic solid mixtures, lyophilized powders, and solid interpenetrating networks. In other embodiments, the molecules are in liquid-comprising forms, such as solutions, emulsions, colloidal suspensions, slurries, or gel mixtures such as hydrogels. In a preferred embodiment, the drug is provided in a solid form, particularly for purposes of maintaining or extending the stability of the drug over a commercially and medically useful time, e.g., during storage in a drug delivery device until the drug needs to be administered. The solid drug matrix may be in pure form or in the form of solid particles of another material in which the drug is contained, suspended, or dispersed. In one embodiment, the drug is formulated with an excipient material that is useful for accelerating release, e.g., a water-swellable material that can aid in pushing the drug out of the reservoir.

The drug can comprise small molecules, large (i.e., macro-) molecules, or a combination thereof. In one embodiment, the large molecule drug is a protein or a peptide. In various other embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants (e.g., LMWH, pentasaccharides), antibiotics (e.g., immunosuppressants), analgesic agents, and vitamins. Examples of suitable types of proteins include, glycoproteins, enzymes (e.g., proteolytic enzymes), hormones or other analogs (e.g., LHRH, steroids, corticosteroids, growth factors), antibodies (e.g., anti-VEGF antibodies, tumor necrosis factor inhibitors), cytokines (e.g., α-, β-, or γ-interferons), interleukins (e.g., IL-2, IL-10), and diabetes/obesity-related therapeutics (e.g., insulin, exenatide, PYY, GLP-1 and its analogs). The drug may be a gonadotropin-releasing (LHRH) hormone analog, such as leuprolide. The drug may be a parathyroid hormone, such as a human parathyroid hormone or its analogs, e.g., hPTH(1-84) or hPTH(1-34). The drug may be selected from nucleosides, nucleotides, aptamers, and analogs and conjugates thereof. The drug may be a peptide with natriuretic activity, such as atrial natriuretic peptide (ANP), B-type (or brain) natriuretic peptide (BNP), C-type natriuretic peptide (CNP), or dendroaspis natriuretic peptide (DNP). The drug may be selected from diuretics, vasodilators, inotropic agents, anti-arrhythmic agents, Ca⁺ channel blocking agents, anti-adrenergics/ sympatholytics, and renin angiotensin system antagonists. The drug may be a VEGF inhibitor, VEGF antibody, VEGF antibody fragment, or another anti-angiogenic agent. The drug may be a prostaglandin, a prostacyclin, or another drug effective in the treatment of peripheral vascular disease.

In one embodiment, the drug is an angiogenic agent, such as VEGF or possibly bone morphogenic protein (BMP). In another embodiment, the drug is an anti-inflammatory, such as dexamethasone and pimecrolimus. The device may release both angiogenic agents and anti-inflammatory agents. In a further embodiment, the drug is a growth factor known in the art for chondrogenesis, including fibroblast growth factor (FGF), insulin-like growth factor (IGF), transforming growth factor beta (TGB-β). In a preferred embodiment, the device releases at least one bone growth promoter. As used herein, the term “bone growth promoter” refers to and includes growth factors, FGF, IGF, PDGF, as well as parathyroid hormone.

Different surface areas or parts of the prosthetic implant device may have different numbers, sizes, and densities of reservoirs from other areas or parts of the device, and different reservoirs can be loaded with different drugs and/or different formulations have different release kinetics from other reservoirs. Such various strategies can be used to obtain complex release profiles in a single device, tailored for a particular indication or patient. The reservoirs in one device can include a single drug or a combination of two or more drugs, and can further include one or more pharmaceutically acceptable carriers (i.e., excipients). Two or more drugs can be stored together and released from the same one or more reservoirs or they can each be stored in and released from different reservoirs. The reservoir optionally could include two (or more) different doses or formulations of the same drug.

The release system may provide a temporally modulated release profile (e.g., pulsatile release) when time variation in plasma levels is desired or a more continuous or consistent release profile when a constant plasma level is needed to enhance a therapeutic effect, for example. When a local exposure is required, the release system may also be placed in the desired therapeutic location and provide either a pulsatile or continuous release profile in a local region. Pulsatile release can be achieved from an individual reservoir, from a plurality of reservoirs, or a combination thereof. For example, where each reservoir provides only a single pulse, multiple pulses (i.e., pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In addition, releasing several pulses of molecules in rapid succession can approximate continuous release. The active release systems described herein can be used alone or on combination with passive release systems, for example, as described in U.S. Pat. No. 5,797,898. For example, the reservoir cap can be removed by active means to expose a passive release system, or a given substrate can include both passive and active release reservoirs.

In various embodiments, the release system further includes one or more matrix materials. In one example, the matrix material comprises one or more synthetic polymers. In another example, the one or more matrix materials comprise a biodegradable, bioerodible, water-soluble, or water-swellable matrix material. In one embodiment, the therapeutic or prophylactic agent is distributed in the matrix material and the matrix material degrades or dissolves in vivo to controllably release the therapeutic or prophylactic agent. The therapeutic or prophylactic agent may be heterogeneously distributed in the reservoir or may be homogeneously distributed in the reservoir.

Different therapeutic or prophylactic agents, or different doses, can be delivered from a single device, either from the same surface region or from different surface regions. In one embodiment, the quantity of therapeutic or prophylactic agent provided for release from at least a first of the reservoirs is different from the quantity of the therapeutic or prophylactic agent provided for release from at least a second of the reservoirs. In another embodiment, the time of release of one of the therapeutic or prophylactic agents from at least a first of the reservoirs is different from the time of release of the therapeutic or prophylactic agent from at least a second of the reservoirs. In one embodiment, a first therapeutic or prophylactic agent is in at least one of the reservoirs and a second therapeutic or prophylactic agent is in at least one other of the reservoirs, the first therapeutic or prophylactic agent and the second therapeutic or prophylactic agent being different in kind or dose.

In a preferred embodiment, the one or more release system is provided in two or more layers having different compositions. In one example, each of the at least two reservoirs comprises at least two layers which comprise the one or more therapeutic or prophylactic agents and at least one layer of a degradable or dissolvable matrix material which does not comprise the one or more therapeutic or prophylactic agents. In another example, at least a first therapeutic or prophylactic agent is contained in a first layer of the two or more layers, and wherein a second therapeutic or prophylactic agent is contained in a second layer of the two or more layers. In another embodiment, multiple layers having different compositions are used, and the different layers all contain a drug, which can be the same or different among the layers. In one embodiment, the drug formulation within a reservoir comprises layers of a drug or drugs and a non-drug material, wherein the multiple layers provide pulsed drug release due to the intervening layers of non-drug. Such a strategy can be used to obtain complex release profiles. Similarly, the technique may be used to release two different drugs that are incompatible with one another or otherwise should not be released at the same time. For example, the layer structure could be Non-Drug/Drug A/Non-Drug/Drug B.

In some embodiments, the drug is formulated as a sustained or controlled release formulation. There are numerous sustained release materials available for preparing compositions of this invention. Exemplary materials include synthetic polymers, such as PLGA, PEG, PLLA, and/or naturally occurring polymers such as hyaluronic acid, chitosan, and alginate. The natural-occurring polymers may or may not be crosslinked by methods known to the art. The polymers may or may not contain a ceramic component, such as tricalcium phosphate or another resorbable, biocompatible, calcium phosphate material such as hydroxyapatite. These materials are intended not only to provide a matrix for sustained/controlled release of a drug, but also to facilitate cellular migration into the porous space of the formulation, which in turn may facilitate bone ingrowth, stabilization, and eventual remodeling of the formulation. In addition, a matrix or tissue scaffolding material may be included as part of the device solely for use in simulating the extracellular matrix, which may foster cell adhesion, migration, proliferation, and/or differentiation at the site of implantation. Possible matrix materials known in the art that may suitable for use with the present drug delivery implants, particularly for dental applications, include calcified or decalcified freeze-dried bone, hydroxyapatite (e.g., OSTEOGEN™), bovine-derived hydroxyapatite, tricalcium phosphate, collagen, hard tissue replacement polymers (e.g., BIOPLANT™) bioactive glass (e.g., PERIOGLAS™), coral-derived calcium carbonate, PLGA, methylcellulose, chitosan, hyaluronic acid ester, and enamel matrix derivative.

Reservoir Caps

In an optional embodiment, the device further includes reservoir caps. A reservoir cap is a discrete structure (e.g., a membrane or thin film) positioned over or disposed in (thereby blocking) the opening of a reservoir to separate the (other) contents of the reservoir from the environment outside of the reservoir. It controls, alone or in combination with the release system, the time and/or rate of release of the therapeutic or prophylactic agent from the reservoir. For example, release can be controlled by selecting which reservoir caps, how many reservoir caps, and at what time the reservoir caps are disintegrated or made permeable. In one embodiment, the reservoir caps are non-porous and have a thickness between 0.1 and 100 microns.

The reservoir caps may be disintegrated in vivo by active or passive means. Any combination of passive or active reservoir caps can be present in a single device. As used herein, the term “disintegrate” is used broadly to include without limitation degrading, dissolving, rupturing, fracturing or some other form of mechanical failure, as well as fracture and/or loss of structural integrity of the reservoir cap due to a chemical reaction or phase change (e.g., melting or vaporization), unless a specific one of these mechanisms is indicated. Examples of suitable reservoir cap opening technologies and the activation means therefor are described in U.S. Pat. No. 5,797,898, U.S. Pat. No. 6,527,762, and U.S. Pat. No. 6,491,666, U.S. Patent Application Publications No. 2004/0121486, No. 2002/0107470 A1, No. 2002/0072784 A1, No. 2002/0138067 A1, No. 2002/0151776 A1, No. 2002/0099359 A1, No. 2002/0187260 A1, and No. 2003/0010808 A1; PCT WO 2004/022033 A2. PCT WO 2004/026281; and U.S. Pat. Nos. 5,797,898; 6,123,861; and 6,527,762, all of which are incorporated by reference herein.

In a preferred embodiment, a discrete reservoir cap completely covers/plugs a single reservoir opening. In another embodiment, a discrete reservoir cap covers two or more, but less than all, openings in a single reservoir. For instance, a single reservoir may have multiple, adjacent openings, in the same surface or side of the device body. See, e.g., U.S. Application No. 2006/0057737 to Santini Jr. et al., which is incorporated herein by reference.

In devices where release is passively controlled, the reservoir caps are formed from a material or mixture of materials that degrade, dissolve, or disintegrate over time, or that do not degrade, dissolve, or disintegrate, but are permeable or become permeable to the therapeutic or prophylactic agent. Representative examples of reservoir cap materials include polymeric materials and various types of semi-permeable membranes, and non-polymeric materials such as porous forms of metals (e.g., trabecular metal, a porous tantalum), semiconductors, and ceramics. Passive semiconductor barrier layer materials include nanoporous or microporous silicon membranes. In one embodiment, the reservoir cap material may be a porous silicon, such as a nanoporous silicon membrane (e.g., NANOGATE™ by Imedd Inc. or a nanostructured porous silicon (e.g., BIOSILICON™ by Psividia Ltd.) NANOGATE™ is used as a non-degradable drug diffusion membrane, whereas BIOSILICON™ is used as a degradable matrix to release drug. In a preferred embodiment, the reservoir caps are non-porous and are formed of a bioerodible or biodegradable material, known in the art, such as a synthetic polymer, e.g., a polyester (such as PLGA), a poly(anhydride), or a polycaprolactone.

In devices where release is actively controlled, the reservoir cap includes any material that can be disintegrated or permeabilized in response to an applied stimulus (e.g., electric field or current, magnetic field, change in pH, or by thermal, chemical, electrochemical, or mechanical means). Electrothermal ablation is a preferred form of active disintegration, as taught in U.S. Patent Application Publication No. 2004/0121486 A1 to Uhland, et al. In other embodiments, the disintegration comprises corrosion, e.g., electrochemically induced oxidation and dissolution. Examples of suitable reservoir cap materials include gold, titanium, platinum, tin, silver, copper, zinc. alloys, and eutectic materials such as gold-silicon and gold-tin eutectics. In various embodiments, the reservoir caps are electrically conductive. In one embodiment, the reservoir caps are in the form of a non-porous, thin metal film. In another embodiment, the reservoir caps are made of multiple metal layers, such as a multi-layer/laminate structure of platinum/titanium/platinum. For example, the top and bottom layers could be selected for adhesion layers on (typically only over a portion of) the reservoir caps to ensure that the reservoir caps adhere to/bonds with both the substrate area around the reservoir openings, reservoir cap supports, and a dielectric overlayer. In one specific example, the structure is titanium/platinum/titanium/platinum/titanium, where the top and bottom layers serve as adhesion layers, and the platinum layers provide extra stability/biostability and protection to the main, central titanium layer. The thickness of these layers could be, for example, about 300 nm for the central titanium layer, about 40 nm for each of the platinum layers, and between about 10 and 15 nm for the adhesion titanium layers. See, e.g., Prescott et al., Nature Biotechnology (12 Mar. 2006); Maloney, et al., J Controlled Release 109:244-55 (2005).

Infection Control

In another aspect, an improved temporary implant is provided for use in controlling infection and/or inflammation at a surgical site, such as the hip or other joint following replacement with a prosthetic implant. The device also may be highly useful in craniomaxillofacial surgery to repair a traumatic facial injury or congenital defect. One particular advantage of this device is that it enables the targeted and sustained local delivery of the drug, and the local delivery advantageously may decrease undesirable systemic drug exposure and deleterious side effects caused thereby. The use of local (as opposed to systemic) antibiotics may also advantageously decrease the dosage requirement to obtain a similar effect on the local environment. That is, upon delivery the drug is concentrated in a local space where it is most needed, and although will eventually reach systemic circulation, it will do so in lower amounts/concentrations than would occur if a therapeutically effective amount of the drug were originally administered by a systemic route, e.g., intravenously or orally.

In one embodiment, this implantable infection control device includes a plurality of beads tethered together to form a chain, wherein the beads comprise a plurality of discrete reservoirs which are loaded with a release system which includes at least one anti-infective drug formulation for controlled release in vivo. The beads may be cylindrical, spherical, or elliptical shaped, or in other shapes, which may be designed to optimize implantation, local drug delivery, or explantation. The beads may be formed from a variety of biocompatible materials. Representative examples of materials of construction include polymers, (e.g., polytetrafluoroethylenes, polyesters, silicones), metals, glasses, ceramics, and combinations thereof. The beads may be tethered together by essentially any flexible elongated material, which is biocompatible, non-degradable during use in vivoa, and sufficiently strong to avoid breakage during explantation. For example, the beads may be tethered together by at least one biocompatible string imbedded through the beads or threaded through apertures in the beads. In various embodiments, the beads are tethered with a string. The “string” can be flexible, biocompatible material, such a nylon thread, braided or unbraided metal or polymer fibers, or the like. Alternatively, multiple separate strings can be used to connect two adjacent beads. The implant device may consist of essentially any number of beads, and the number would be selected based for example on the size of the beads, the drug release kinetics desired and provided by each bead, the area over which the drug is to be locally released, and other factors. In a preferred embodiment, the device would have at least five beads and no more than 100 beads tethered together (e.g., between ten and fifty beads). Multiple, unconnected chains may be implanted together. In one embodiment, the diameter of the beads is between about 2 and about 10 mm, e.g., between 4 and 8 mm.

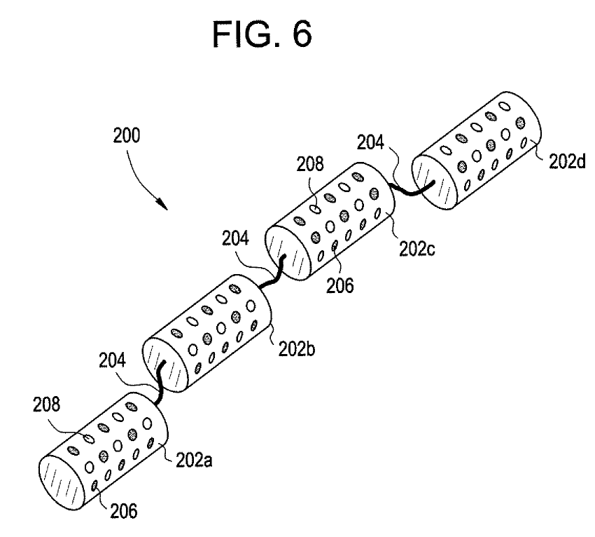

One example of the implantable infection control device is illustrated in FIG. 6. Chain device 200 includes beads 202a, 202b, 202c, and 202d, which are tethered together by string 204. Each bead includes a plurality of reservoirs loaded with drug formulation. In this example, reservoirs 208 alternate with reservoirs 206. The alternate reservoirs contain different formulations of the same or different drugs.

In various embodiments, the release system may be tailored to release one, two, or more different drugs. In a preferred embodiment, the release system comprises at least one antibiotic agent dispersed in a polymeric matrix material. One can control the amount and rate of drug released from a device by selecting a particular composition of the drug formulation and by varying the number, size, and placement of the reservoirs. Release rate can be tailored, for example, by including a biodegradable or bioerodible matrix material as known in the art.

In another embodiment, a first group of the reservoirs in the beads comprises the at least one anti-infective drug formulation and a second group of the reservoirs comprises a second formulation of a drug, wherein the at least one anti-infective drug formulation and the second formulation have different compositions. The drug of the at least one anti-infective drug formulation may be different from the drug of the second formulation, or the drug may be the same but formulated in different dosages, e.g., to release at different times or different rates. In one embodiment, the second formulation of the drug comprises an anti-inflammatory agent. In another embodiment, the first formulation comprises a first antibiotic and the second formulation comprises a different an antibiotic. By selecting different formulations and structures of the release system, the device can be adapted to provide simultaneous release of the two or more drugs, serial release of two or more drugs, or other release profiles. In one example, the release system may be layered in the reservoirs to provide serial release of two or more drugs. For example, a layer of a therapeutic agent and a layer of inactive material could be alternately loaded into the reservoirs, or formulations having different concentrations could be stacked to provide a drug concentration gradient. The layers may be solid or porous. These techniques and variations thereof can be used to create different and complex profiles of drug release (constant, pulsatile (on/off) sinusoidal, short burst of high dose followed by constant low dose, etc.), which may be desirable depending upon the particular therapeutic applications and beneficial agents being delivered. For ease of manufacture, each bead may contain only a single drug, but beads containing different drugs may be connected together (e.g., in an alternating manner) to yield a multidrug device, where each bead releases only one drug. This technique may be easier to implement than producing beads having multiple drugs in each bead.

Manufacture of these devices should be relatively easy/inexpensive to implement using standard polymer forming methods, such as injection molding, stamping, therocompression molding, and/or extrusion coupled with subsequent drilling to form the reservoirs in the extruded device.

In another aspect, revision implants are provided with reservoirs on the implant surface or in crevices or channels, which are loaded with a stable antibiotic formulation with optimized release kinetics. See the device of FIG. 1 for an example of a possible device structure. In this way, the antibiotic agent (e.g., gentamicin) can be released under a bacterial biofllm that may form from bacteria harbored in crevices of a prosthetic implant.

Osteonecrosis Treatment Device and Method

In another aspect, devices and methods are provided for use in the treatment of osteonecrosis, such as osteonecrosis of the femoral head. In one embodiment, the method for treating osteonecrosis includes the steps of: (a) removing necrotic bone tissue from a bone and creating one or more channels or voids in said bone; and (b) inserting at least one drug delivery device into the one or more channels or voids, wherein the drug delivery device comprises a body portion in which are provided a plurality of discrete reservoirs containing at least one release system comprising one or more therapeutic or prophylactic agents for release in vivo. Following insertion of the device, the remaining open space in the channel optionally may be “back-filled” with a suitable back-fill material, such as a bone graft material, cement, or polymeric material, which are known in the art. The drug delivery device advantageously provides greater control of release kinetics and offers structural support that may not be provided if using a back-fill material alone. It is important that the device, with or without the backfill material, provides sufficient mechanical/structural support to the surface of the femoral head.

In one embodiment, the device releases a bone resorption inhibitor to keep necrotic bone tissue from disappearing to rapidly—when left purposefully by the surgeon for structural purposes—while new bone is forming. Preferably, the device would release both the bone resorption inhibitor and a bone growth factor in therapeutically effect amounts to achieve beneficial rates of loss of necrotic tissue and of new bone growth. See, e.g., Shanbhag, et al., Clinical Orthopaedics & Related Res, 344:33-43 (1997); Shanbhag, et al., “Biological Response to Wear Debris: Cellular Interactions Causing Osteolysis” in The Adult Hip (Callahan, et al., eds.) (Lippincott-Raven Williams, N.Y. 2006).

The body of the device (i.e., the substrate) can be made of a bone graft material (autograft, allograft, etc.), a suitable resorbable polymeric material, a metal, or a combination thereof. Such materials are known in the art. The body portion may be a monolithic structure, or may include two or more segments that can be placed together, e.g., in a close or fitting arrangement, at the site of implantation.

In a preferred embodiment, the release system comprises a drug selected from growth factors (e.g., BMPs), angiogenesis promoters, or combinations thereof.

Multiple devices can be inserted, e.g., stacked, into a single channel if desired, for ease of insertion or for tailoring release of drug over a greater local area within the bone. In one variation of this method, two or more of the drug delivery devices are inserted into a single channel. In another variation of the method, multiple channels are formed in the same bone, and one or more of the drug delivery devices are inserted into the multiple channels. The channels may be substantially parallel and near each other.

In one embodiment, the method further includes the step of including/using a fluid delivery means to wet the at least one drug delivery device disposed in the one or more channels or voids. The fluid delivery means may be in the form of a re-routed or grafted blood vessel, to direct blood into the channel or void. This technique, where a portion of a patient's own fibula is used as the graft, is known as “vascularized fibular grafting” in current practice. Alternatively, the fluid delivery means may include an external fluid source, a pump, and at least one catheter having a proximate end and a distal end, wherein the distal end of the catheter is inserted into at least one of the channels or voids containing the drug delivery device and delivers fluid from the fluid source via the pump. By delivering a biocompatible fluid into one or more bone channels following insertion of the drug delivery implant device, tissue regrowth into the channel is promoted by providing a suitably “moist” environment in the otherwise relatively dry channel so that drug release/diffusion from the prosthetic device can occur and be effective/viable. The precise location of the distal end of the catheter can be adjusted depending upon the needs of the patient and the particular structure of the implant device. It is noted that the catheter is temporary and would be removed once the physician determined that the osteonecrotic lesion had begun to heal. In one embodiment, the fluid delivery means is a surgically relocated blood vessel (e.g., a blood vessel graft, as described in Aldridge & Urbaniak, “Bone Grafting for Osteonecrosis of the Femoral Head” Seminars in Arthroplasty, pp. 151-60 (Elsevier Inc. 2004)). In another embodiment, the fluid delivery means includes a mechanical pump system. For instance, the pump system may include a pump and catheter for delivering saline, whole blood, plasma, or another blood component, or the like, from a fluid supply which is located externally to the patient. External drug pumps are easily accessible and are in medical use in other therapeutic applications, such as the externally worn insulin pump. In one embodiment, hyaluronic acid is included in the fluid. The fluid would be supplied for a period effective to promote drug release/tissue regeneration, after which time the catheter can be removed. This approach may allow the patient to be ambulatory without the risk that physical activity could cause damage to a vascular graft and consequent internal bleeding.

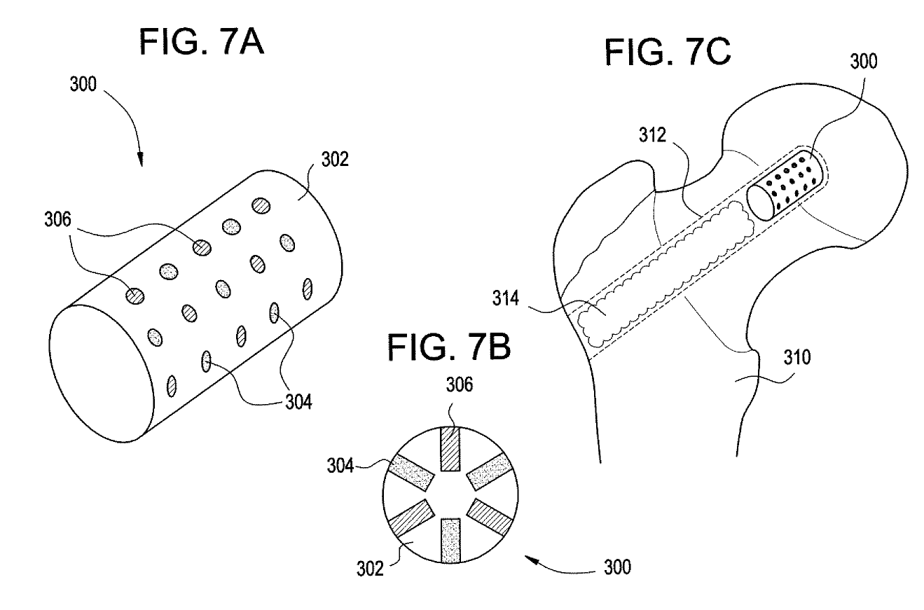

A method, similar to core decompression, has been developed for extending the effective life of the bone tissue (e.g., in a patient exhibiting early stages of osteonecrosis), in particular the tissue of the femoral head, wherein a channel is made in the femoral head and a reservoir-containing drug delivery device is inserted into the channel. Some or all of the necrotic bone is removed during the creation of the channel. One embodiment is illustrated in FIGS. 7A-C. The device 300 is shaped to fit into a channel 312 in the femoral head of femur 310, provide (at least temporary) structural support, and release one or more therapeutic agents to promote the vascularization and growth of healthy bone tissue in the femoral head. Some of the reservoirs 304 are loaded with a release system comprising one or more BMPs (e.g., OP-1, BMP-2) and/or other reservoirs 306 are loaded with a release system comprising one or more angiogenesis promoters (e.g., VEGF, FGF). Following insertion of the device, the remaining open space in the channel is back-filled with a suitable back-fill material 314. The backfill material may be a matrix material as described above to enhance cellular adhesion, or may be a packed, ground bone graft, demineralized bone matrix, a biocompatible polymer or cement, or the like as known in the art.

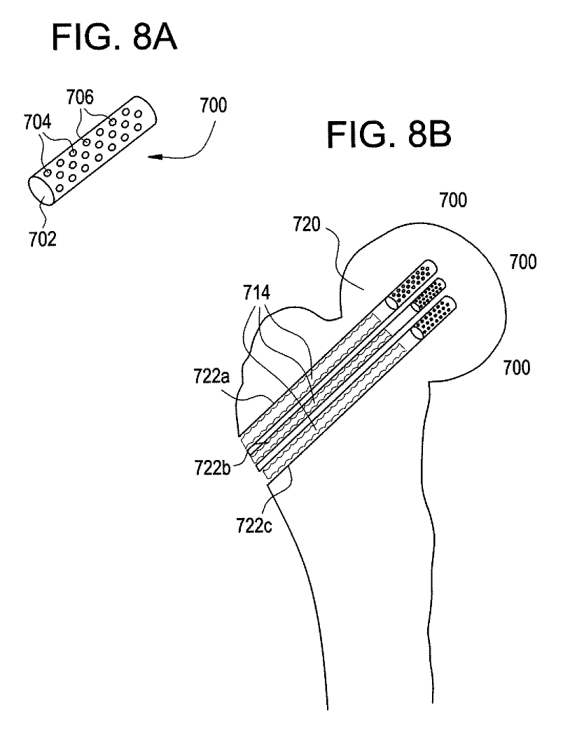

In another embodiment the core decompression is performed creating multiple smaller diameter channels, rather than one single large diameter channel, which may be desirable as a treatment method for certain lesion sizes. Furthermore, this approach may reduce the risk of weakening and fracture of the femoral neck, a potential and serious complication that may occur with the use of a single large channel. In a representative example, shown in FIG. 8, devices 700 are shaped to fit in the channels 722a, 722b, and 722c in femoral head 720. Each device 700 has an elongated cylindrical body 702. The body 702 includes an array of multiple discrete, defined reservoirs, which are loaded with first drug formulation 704 and second drug formulation 706, that contain one or more formulations or therapeutics, substantially as described in the single channel treatment embodiment. Following insertion of the devices 700, the channels 722a,b,c can be back-filled with a bone graft material, cement, or polymeric material 714.

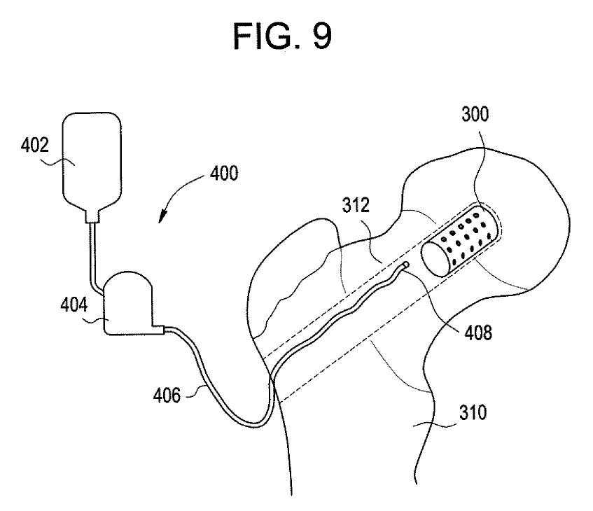

Another embodiment is shown in FIG. 9, which illustrates a reservoir-based drug delivery implant 300 installed into an insert channel 312 in the femoral head of femur 310 of a patient. A fluid delivery system 400 is provided: The distal end 408 of a catheter 406 is also placed into the insert channel, and the proximal end of the catheter is connected to a metering pump 404, which is connected to fluid supply 402. Following insertion of the device and catheter, the channel can be back-filled with a suitable material (not shown). In one embodiment, the pump delivers a steady, small amount of a sterile, biocompatible fluid to keep the channel moist and providing a medium through which drug from the rigid implant 300 can diffuse to promote bone tissue healing/growth. In another embodiment, the pump and the fluid supply (e.g., a fluid reservoir) can be integrated into a single unit, a single device.

In one embodiment, the drug delivery implant is itself provided with one or more channels to accommodate passage of the catheter through, or by, the drug delivery implant. For example, the device may include an axial exterior or central channel, such that the catheter can pass by or through the device to deliver fluid on the distal side of the implant. Such a through-channel would also allow perfusion of the device with the fluid, particularly if the device is at least partially permeable/porous.

In another embodiment for the treatment of avascular necrosis, the drug delivery implant is a drug containing material, wherein the drug is provided in other than discrete reservoir form. In one case, the implant channel is loaded with a drug containing material is in the form of granules of tricalcium phosphate that have been compressed together to form a unitary device. See U.S. Patent Application Publication 2005/0170012, which is incorporated herein by reference and which describes tricalcium phosphate composition for applications other than treatment of avascular necrosis. Alternatively, the implant channel is loaded with another material that is another rigid porous matrix or soft (non-rigid) biomaterial known in the art, e.g., a bone cement, that has been loaded with (e.g., homogeneously mixed with) one or more drugs. In various embodiments, combinations of rigid implants and non-rigid backfill or cement materials can be used to delivery one or more drugs into an insert channel created in bone tissue, with or without an allograft. Multiple devices can be inserted, e.g., stacked, into a single channel if desired, for ease of insertion or for tailoring release of drug over a greater local area within the bone. Following insertion of the device and catheter, the channel can be back-filled with a bone graft material, cement, or polymeric material. In such an embodiment, the catheter preferably includes a polytetrafluoroethylene coating, so that the backfill material does not adhere to it, thereby permitting catheter removal at a later time.

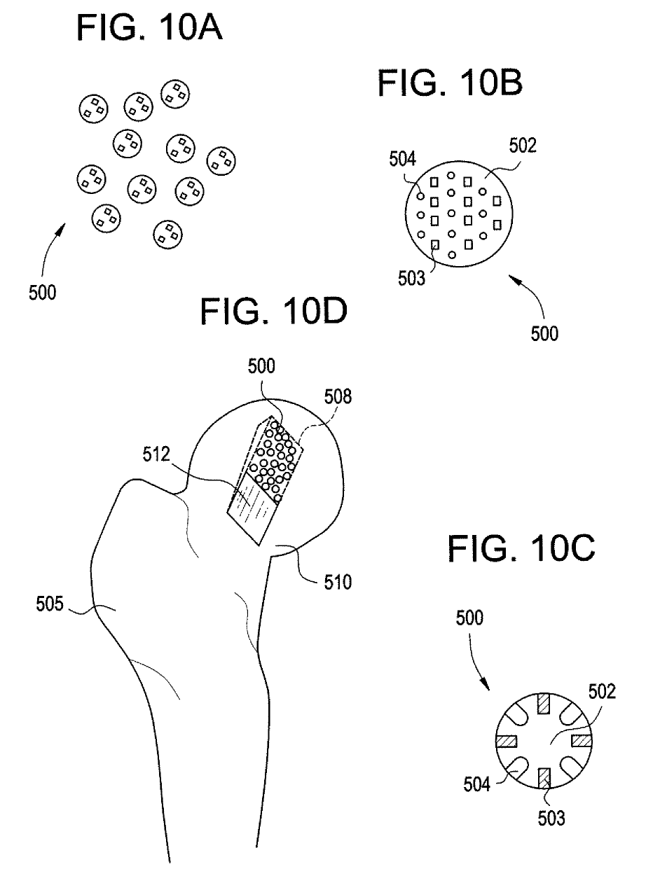

In one embodiment, an operative technique known as the “light bulb procedure” for treating osteonecrosis can be modified for use with the methods and devices described herein. The procedure is a type of non-vascularized bone grafting. In one technique, which is show in FIGS. 10A-D, an opening/window 512 is created in femur 505 at the base of the head 510. The necrotic tissue is removed, and the remaining void 508 is then packed with beads 500. (The beads are shown as spherical, but could be in other shapes and forms, such as granules, cylinders, etc.). Each bead includes a body 502 having a surface in/on which are arrayed multiple discrete reservoirs, which contain at least two different release systems 503 and 504. These release systems could be two different therapeutic agents or a single therapeutic agent in two different formulations to achieve two different release profiles. For instance, the release systems may include one or more growth factors, angiogenic factors, antibiotics, other therapeutic agents, or a combination of different therapeutic agents. The characteristics of release of these therapeutic agents may be dictated, at least in part, by the chemical composition and physical state of the release system. For example, the release system may be or include a lyophilized solid, lyophilized solid in a solid matrix, a gel/hydrogel formulation, a growth factor-loaded porous sponge, a polymer matrix, or the like. In one case, the therapeutic agent in release system 503 or 504 is a growth factor, preferably BMP-2. In another case, the therapeutic agent in release system 503 or 504 is an angiogenic factor, preferably FGF. In still another case, a combination of two or more therapeutic agents are used, such as BMP-2 in release system 503 together with FGF in release system 504.

In an alternative embodiment, the beads do not include built-in discrete reservoirs. In which case, the channel may be packed with microspheres or nanospheres to deliver drug and provide some mechanical support. For example, the microspheres could be a controlled release microsphere as known in the art. For instance, the microsphere could be formed of a biodegradable or non-degradable polymeric material having an encapsulated or layered drug therein. In one case, the microsphere may be in the form of a double-walled structure as made by Spherics, Inc. See, e.g., U.S. Pat. No. 6,531,154 to Mathiowitz et al., U.S. Pat. No. 5,912,017 to Mathiowitz et al., which are incorporated herein by reference.

The bead body may be composed of a resorbable ceramic, such as tricalcium phosphate or other biocompatible calcium phosphates, polymers, or a composite of these. The greater surface area of the multiple beads advantageously would allow for higher drug per volume exposure, and while currently cancellous bone (often autograft) is used to pack the void, by using these beads, there would be no need to harvest bone from a secondary site. The interstitial space between the beads and created by the reservoirs themselves may also enhance bone ingrowth to eventually fill the void with newly remodeled bone. In another embodiment, the beads may be packed into the space with a fluid or other “carrier” which may or may not “set” to form a gel/hydrogel upon reaching body temperature (nominally 37° C.) to provide for further physical stabilization within the void as well as provide a moist environment to promote the drug release into the space. In one embodiment, the fluid or gel may contain hyaluronic acid, a component of synovial fluid. This “carrier” may also gently expand upon setting in order to completely fill the void and provide for further increased stabilization.

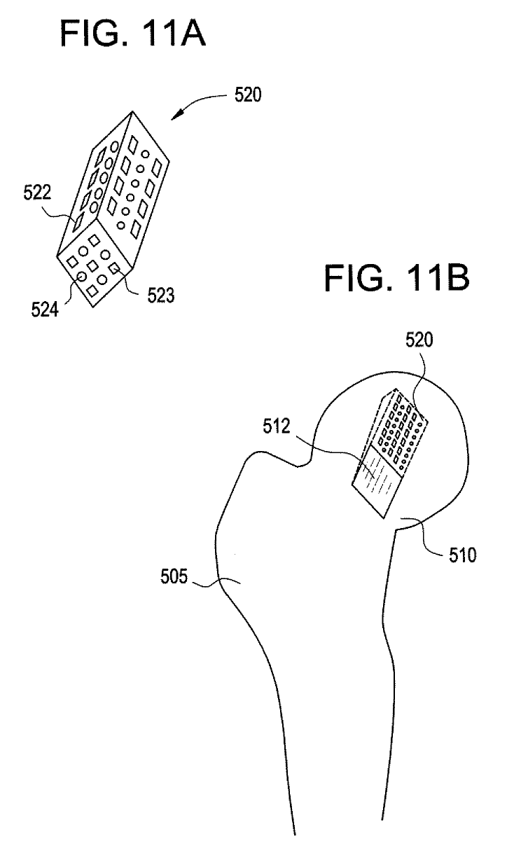

In one variation, instead of multiple beads, a single device is used, which is molded (or otherwise fabricated) to fit neatly into the space created by debridement of the femoral head, especially in cases where the head may be in danger of surrounding tissue collapse due to defect size and location, and enhanced stabilization of the void is highly desired. This embodiment is illustrated in FIGS. 11A-B, which shows implant device 520, having device body 522 which includes arrays of discrete reservoirs containing first drug formulation 523 and second drug formulation 524. In other embodiments, one or three or more different drug formulations may be used.) Optionally, the surface of the device 520 prior to insertion may be coated with a fluid or gel that will provide a moist environment and path for drug release contacting the remaining femoral head tissue inside the debrided region. Alternatively or in addition, a fluid delivery system (described above) may be used with either of these light bulb procedures.

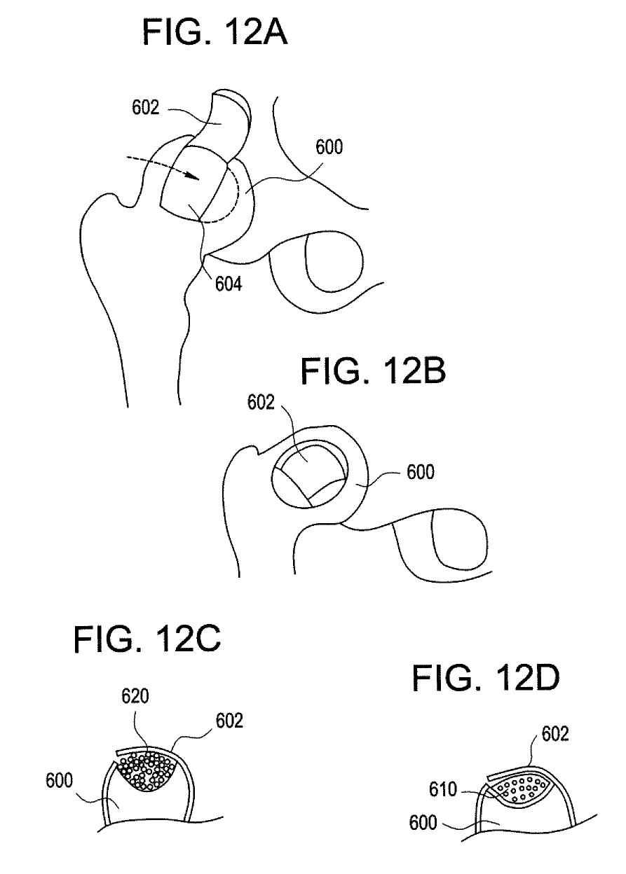

In another embodiment an operative technique known as the “trap-door procedure can be modified for use with the methods and devices described herein. The procedure is another type of non-vascularized bone grafting. In one technique, which is shown in FIGS. 12A-D, an osteochrondral flap 602 is opened in the femoral head 600 and the lesion area is debrided, leaving a void space 604. Similarly to the embodiments described by FIGS. 10-11, the void is filled with either beads 620, or rigid device 610 shaped to fit the void space 604. Then, the osteochrondral flap 602 is closed over the beads or device. The beads 620 or device 610 have a plurality of discrete reservoirs in which one or more release systems/drug formulations are incorporated. A fluid delivery system optionally may be included as described above.

Cartilage Engineering and Joint Resurfacing

In another aspect, implant devices are provided to promote the growth of avascular tissue, such as articular cartilage, and extend the longevity of a person's natural cartiiag—e.g., to delay the need for a total knee or hip replacement. In one embodiment, a reservoir-containing drug delivery device is placed in or near the intercondylar fossa, between the condyle, or within/under the synovial sac, and the reservoirs of the device are loaded with a formulation for controlled release of one or more growth factors (FGF, IGF, TGF-β, etc.) to promote chondrogenesis. The device body (substrate) can be shaped and sized to fit near, and provide local drug release to, the cartilage without interfering with movement of the joint.

In another embodiment, devices and methods are provided for use in joint resurfacing. For example, in a conventional resurfacing system, a metal cap is placed over the end of an articular surface to extend the useful life of a failing joint. The present improvement provides a cap having a plurality of discrete reservoirs for releasing growth factors or other therapeutic agents to promote chondrogenesis.

In a preferred embodiment, a joint resurfacing device is provided that includes a body portion having ajoint tissue interfacing surface and an opposing side; a plurality of discrete reservoirs located in or on the joint tissue interfacing surface; at least one release system disposed in one or more of the plurality of reservoirs containing at least one release system comprising one or more therapeutic or prophylactic agents for release in vivo; and an anchor portion extending from the opposing side away from the joint tissue interfacing surface, wherein the anchoring portion is adapted to secure the joint resurfacing device to a bone in need of resurfacing. In various embodiments, the one or more therapeutic or prophylactic agents are selected from growth factors (e.g., BMPs), angiogenesis promoters, analgesics, anesthetics, antibiotics, and combinations thereof. In a preferred embodiment, the therapeutic agent is a growth factor to promote chondrogenesis.

In a preferred embodiment, the joint tissue interfacing surface comprises a rounded cap, and the reservoirs have chamfered openings in the surface of the joint tissue interfacing surface. The anchor portion may be in the form of a rod or screw. The body portion may be formed of a metal (e.g., a titanium alloy, a cobalt chromium alloy, or a cobalt chromium molybdenum alloy), a polymer (e.g., an ultra high molecular weight polyethylene), a ceramic, or a combination thereof. In one variation, the device consists of a monolithic metal structure.

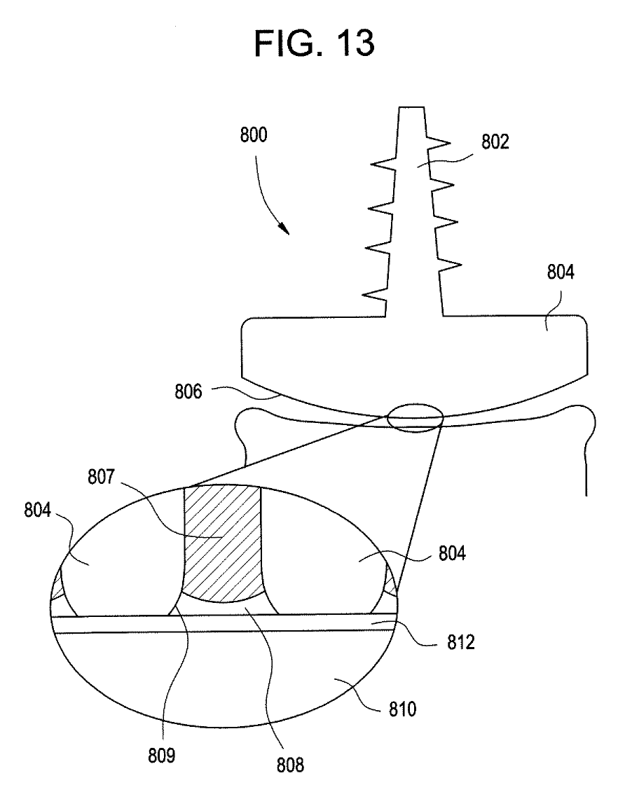

One embodiment of such a device is shown in FIG. 13. Resurfacing device 800 includes a main body portion 804 and an anchoring portion 802. The body portion 804 includes a surface 806 that contacts the repaired/reinforced intra-articular cartilage 812 on bone 810. Surface 806 includes a plurality of discrete reservoirs 808, which are loaded with a release system 807 that includes a growth factor and/or other therapeutic or prophylactic agents. The reservoirs 808 have an opening with smooth, rounded edges 809 to minimize frictional engagement with the surface of the adjacent cartilage 812. In another embodiment, following a total knee replacement, the prosthetic knee device includes a plurality of discrete reservoirs for releasing an antibiotic or other drug.

Dental Devices

In one embodiment, a dental prosthetic device is provided that includes an anchor portion for anchoring in a bone structure (e.g., a jaw bone) and a head intended to support a dental prosthesis, and reservoirs are provided in one or more parts, preferably at the anchor portion. Typically, the reservoirs deliver one or more drugs locally at the implant site over an extended period of time following implantation. Other dental prosthesis known in the art can be adapted to include the reservoir-based controlled release formulations described herein. See U.S. Pat. No. 6,799,970, which is incorporated herein by reference.

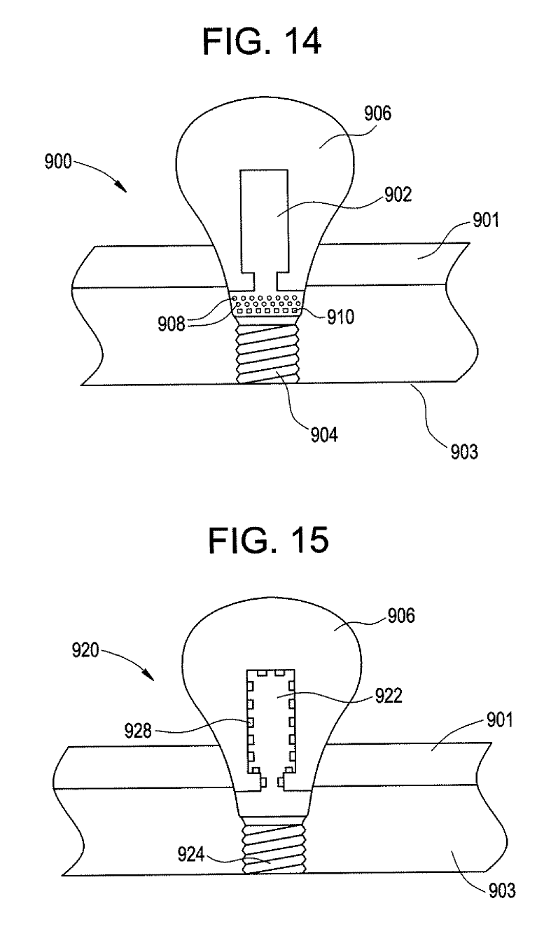

Exemplary, non-limiting embodiments of dental prosthetic devices are illustrated in FIGS. 14-16. These figures illustrate variations of how the multi-reservoir techniques for controlled drug delivery can be adapted to dental implant devices. Further details about dental implant devices are described, for example, in U.S. Pat. No. 6,896,517, U.S. Pat. No. 6,375,465, and U.S. Patent Application Publication No. 2005/0089813, which are incorporated herein by reference.

FIG. 14 shows dental prosthetic device 900 implanted in the jawbone 903 and gum 901 of a patient. The device includes a device body having an anchor portion 904 adapted for engagement with a jawbone of a patient in need thereof and a mounting stem portion 902 (i.e., a post) on which a replacement tooth portion 906 is mounted. The device body includes a first array of discrete reservoirs 908 located in spaced apart positions in the device body, which reservoirs are loaded with a first release system which includes a therapeutic or prophylactic agent for controlled release in vivo. The device body also includes a second array of discrete reservoirs 910 located in spaced apart positions in the device body, which reservoirs are loaded with a second release system which includes the same or a different therapeutic or prophylactic agent for controlled release in vivo.

FIG. 15 shows dental prosthetic device 920 implanted in the jawbone 903 and gum 901 of a patient. The device includes a device body having an anchor portion 924 adapted for engagement with a jawbone of a patient in need thereof and a mounting stem portion 922 on which a replacement tooth portion 906 is mounted. The device body includes a first array of discrete reservoirs 928 located in spaced apart positions in the mounting stem portion. The reservoirs are loaded with a release system which includes a therapeutic or prophylactic agent for controlled release in vivo. In one case, the replacement tooth is not mounted onto the stem until after at least partial healing has occurred at the implant site following implantation of the device body; the drug is released, at least in part, before the replacement tooth is installed onto the stem. In another case, the device could include a channel or other flow path means (not shown) for the therapeutic or prophylactic agent to flow into contact with tissues at the site of implantation.

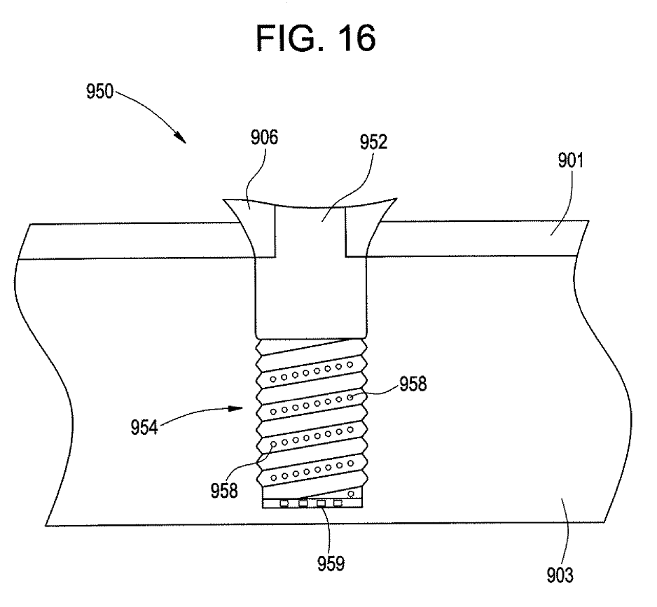

FIG. 16 shows the anchor portion of dental prosthetic device 950 implanted in the jawbone 903 and gum 901 of a patient. The device body 952 includes an anchor portion 954 which includes discrete reservoirs 958 and 959 located in spaced apart positions in the sides and bottom, respectively, of the anchor portion. The reservoirs are loaded with at least one release system which includes a therapeutic or prophylactic agent for controlled release in vivo. The reservoirs may preferably be microreservoirs.

A wide variety of therapeutic, prophylactic, or diagnostic agents can be released. In a preferred embodiment, the device provides local delivery of one or more anti-infective agents, such as antibiotics known in the art. In another embodiment, the dental implant locally delivers one or more growth factors. Combinations of these can be delivered in vivo to different local areas proximate the implant. Representative drug molecules that may be delivered by these devices include transforming growth factor-beta (TGF-β (e.g., TGF-β-1 or TGF-β-2)), laminin/epidermal growth factor (EGF), bone morphogenic protein (BMP), and combinations of factors, BMP, TGF-β, platelet-derived growth factor (PDGF), and basic fibroblast growth factor (bFGF). These drugs may be combined with a controlled release matrix material, such as a biodegradable or bioerodible polymer.

In one embodiment, the dental implant includes one or more sensors. The sensor may be disposed in the device, e.g., in the rod underneath the replacement tooth, or in other regions of the implant. In one embodiment, the sensor is one that measures temperature, which may be indicative of an infection in the tissue at the implantation site. In another embodiment, the sensor may indicate pressures experienced by the prosthetic tooth. Other types of sensors and diagnostic agents may be included in the dental implant device or portions thereof.

In one embodiment, the implant includes an anchor portion, which may be made of a titanium alloy for example. This anchor portion preferably includes a distal threaded portion for securing into the bone of a patient. Reservoirs can be built into the device at the end, between the threads, above the threads, or combinations thereof. Distal the anchor portion, the device may include a mounting portion on which a replacement tooth (e.g., typically a ceramic or porcelain construction) is attached. The mounting portion may be rod-like and may be integral with or attached to the anchor portion.

Publications cited herein are expressly incorporated by reference. Modifications and variations of the methods and devices described herein will be obvious to those skilled in the art from the foregoing detailed description. Such modifications and variations are intended to come within the scope of the appended claims.

We claim:

1. An implantable medical device for use in the treatment of osteonecrosis comprising:
at least one implant device body adapted for insertion into one or more channels or voids in bone tissue;
a plurality of discrete reservoirs located in the surface of the at least one implant device body; and
at least one release system disposed in one or more of the plurality of reservoirs, wherein the release system includes at least one drug selected from the group consisting of bone growth promoters, angiogenesis promoters, analgesics, anesthetics, antibiotics, and combinations thereof.

2. The device of claim 1, wherein the device body is formed of a bone graft.

3. The device of claim 1, wherein the device body is formed of a polymer, a metal, a ceramic, or a combination thereof.

4. The device of claim 1, wherein the device body is cylindrical shaped.

5. The device of claim 1, wherein the device body is in the form of a plurality of beads.

6. The device of claim 1, wherein the discrete reservoirs are microreserviors.

7. A method for treating osteonecrosis comprising the steps of:
removing necrotic bone tissue from a bone and creating one or more channels or voids in said bone; and
inserting at least one drug delivery device into the one or more channels or voids, wherein the drug delivery device comprises a body portion in which are provided a plurality of discrete reservoirs containing at least one release system comprising one or more therapeutic or prophylactic agents for release in vivo.

8. The method of claim 7, wherein the release system comprises a drug selected from bone growth promoters, angiogenesis promoters, or combinations thereof.

9. The method of claim 7, wherein two or more drug delivery devices are inserted into two or more channels formed in said bone.

10. The method of claim 7, further comprising utilizing a fluid delivery means to wet the at least one drug delivery device disposed in the one or more channels or voids.

11. The method of claim 10, wherein the fluid delivery means comprises a re-routed or grafted blood vessel.

12. The method of claim 10, wherein the fluid delivery means comprises a fluid source, a pump, and at least one catheter having a proximate end and a distal end, wherein the distal end of the catheter is inserted into at least one of the channels or voids containing the drug delivery device and delivers fluid from the fluid source via the pump.

13. The method of claim 12, wherein the fluid reservoir and pump are integrated into a single device.

14. The method of claim 12, wherein the fluid source comprises saline, blood, a blood component, hyaluronic acid, or a combination thereof.

15. The method of claim 7, wherein the body portion comprises a bone graft, a polymer, a metal, or a combination thereof

16. The method of claim 7, wherein the body portion is a monolithic structure.

17. The method of claim 7, wherein the body portion is in the form of a plurality of beads.

18. The method of claim 7, wherein the discrete reservoirs are microreservoirs.

19. The method of claim 7, wherein the step of removing necrotic bone tissue from a bone and creating one or more channels or voids in said bone involves a light bulb surgical procedure or trapdoor surgical procedure.

20. A joint resurfacing device comprising:
a body portion having a joint tissue interfacing surface and an opposing side;
a plurality of discrete reservoirs located joint tissue interfacing surface;
at least one release system disposed in one or more of the plurality of reservoirs containing at least one release system comprising one or more therapeutic or prophylactic agents for release in vivo; and
an anchor portion extending from the opposing side away from the joint tissue interfacing surface, wherein the anchoring portion is adapted to secure the joint resurfacing device to a bone in need of resurfacing.

21. The device of claim 20, wherein the one or more therapeutic or prophylactic agents are selected from the group consisting of BMPs, angiogenesis promoters, analgesics, anesthetics, antibiotics, and combinations thereof.

22. The device of claim 20, wherein the one or more therapeutic or prophylactic agents comprises a bone growth promoter.

23. The device of claim 20, wherein the joint tissue interfacing surface comprises a rounded cap.

24. The device of claim 20, wherein the reservoirs have chamfered openings in the surface of the joint tissue interfacing surface.

25. The device of claim 20, wherein the anchor portion comprises at least one screw.

26. An implantable infection control device comprising:
a plurality of beads tethered together to form a chain,
wherein the beads comprise a plurality of discrete reservoirs which are loaded with a release system comprising at least one anti-infective drug formulation for controlled release in vivo.

27. The device of claim 26, wherein the beads are cylindrical, spherical, or elliptical shaped.

28. The device of claim 26, wherein the beads comprise a biocompatible material selected from polytetrafluoroethylenes, polyesters, polymethylmethacrylates, silicones, metals, glasses, ceramics, bone cements, and combinations thereof.

29. The device of claim 26, wherein the release system comprises at least one antibiotic agent dispersed in a polymeric matrix material.

30. The device of claim 26, wherein the beads are tethered by at least one biocompatible string imbedded through the beads or threaded through apertures in the beads.

31. The device of claim 26, wherein a first group of the reservoirs comprises the at least one anti-infective drug formulation and a second group of the reservoirs comprises a second formulation of a drug, wherein the at least one anti-infective drug formulation and the second formulation have different compositions.

32. The device of claim 26, wherein at least one of the beads comprises a first drug and at least another of the beads comprises a second, different drug.

33. The device of claim 31, wherein the drug of the at least one anti-infective drug formulation is different from the drug of the second formulation.

34. The device of claim 33, wherein the second formulation of a drug comprises an anti-inflammatory agent.

35. The device of claim 31, which is adapted to provide simultaneous release of the two or more drugs.

36. The device of claim 31, wherein the release system is layered to provide serial release of two or more drugs.

37. A prosthetic dental device comprising:
a device body having an anchor portion adapted for engagement with a jaw bone of a patient in need thereof;
two or more discrete reservoirs located in spaced apart positions in the device body, the reservoirs formed with an opening at the surface of the device body and extending into the device body; and
a release system disposed in the reservoirs which comprises at least one therapeutic or prophylactic agent,
wherein following implantation into a patient the therapeutic or prophylactic agent is released in a controlled manner from the reservoirs.

38. The device of claim 37, further comprising a replacement tooth portion.

39. The device of claim 37, wherein the reservoirs are located in the anchor portion.

40. The device of claim 37, wherein the discrete reservoirs are microreservoirs.

41. The device of claim 37, wherein the device body comprises a stainless steel, a chrome-cobalt alloy, a titanium alloy, a ceramic, an ultra high molecular weight polyethylene, or a combination thereof.

42. The device of claim 37, wherein the anchor portion comprises at least one screw-like, threaded region.

43. The device of claim 37, wherein the therapeutic or prophylactic agent comprises one or more anti-infective, antibiotic agents, growth factors, or a combination thereof.

44. The device of claim 37, wherein the device is adapted to release two or more, different therapeutic or prophylactic agents.

45. The device of claim 44. wherein one of the therapeutic or prophylactic agents is disposed in a first array of reservoirs and a second of the therapeutic or prophylactic agents is disposed in a second array of reservoirs.

46. The device of claim 45, wherein the first array is located to release one or more anti-infective or antibiotic agents into gum tissues.

47. The device of claim 45, wherein the second array is located to release one or more growth factors into orthopedic tissues.

48. A prosthetic dental device comprising:
a device body having an anchor portion for engagement with a jaw bone of a patient in need thereof; and
at least one sensor or diagnostic agent integrated into the device body.

49. The device of claim 48, wherein the sensor can be used to measure temperature, pressure, or both in one or more areas in or around the site of in vivo implantation of the device.
